(19) United States
(12) Patent Application Publication
(10) Pub. No.: US 2024/0335420 A1
(43) Pub. Date: Oct. 10, 2024

(54) N-ACYLHYDRAZONE COMPOUNDS CAPABLE OF INHIBITING NAV 1.7 AND/OR NAV 1.8, PROCESSES FOR THE PREPARATION THEREOF, COMPOSITIONS, USES, METHODS FOR TREATMENT USING SAME AND KITS

(71) Applicants: EUROFARMA LABORATÓRIOS S.A., São Paulo (BR); Universidade Federal do Rio de Janeiro - UFRJ, Rio de Janeiro (BR)

(72) Inventors: Gabriela BARREIRO, São Paulo (BR); Danilo Pereira De SANT'ANA, São Paulo (BR); Luis Eduardo Reina GAMBA, São Paulo (BR); Carlos Alberto Manssour FRAGA, Rio de Janeiro (BR); Eliezer Jesus de Lacerda BARREIRO, Rio de Janeiro (BR); Lídia Moreira LIMA, Rio de Janeiro (BR)

(21) Appl. No.: 18/294,913

(22) PCT Filed: Aug. 2, 2022
(86) PCT No.: PCT/BR2022/050304

§ 371 Date: Feb. 2, 2024

Related U.S. Application Data
(60) Provisional application No. 63/228,516, filed on Aug. 2, 2021.

(51) Int. Cl. A61K 31/382; A61K 31/341; A61K 31/4458; A61K 31/451; A61P 25/02; C07D 213/53; C07D 213/73; C07D 213/81; C07D 307/68; C07D 409/12

(52) U.S. Cl. CPC A61K 31/382; A61K 31/341; A61K 31/4458; A61K 31/451; A61P 25/02; C07D 213/53; C07D 213/73; C07D 213/81; C07D 307/68; C07D 409/12

(57) ABSTRACT

N-Acylhydrazone compounds that are Nav 1.7 and/or Nav 1.8 inhibitors, including N-acylhydrazone compounds of Formula (I), wherein substituents R1 to R4 are independently selected from the groups defined in the specification, as well as to the processes for the preparation thereof, compositions comprising at least one of these compounds, uses, treatment methods for treating or preventing pain-related pathologies, and kits, useful in the fields of medicinal chemistry, organic synthesis, as well as in the treatment of pain-related disorders.

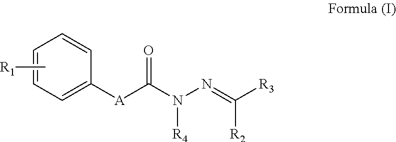

### FIELD OF INVENTION

The present invention relates to N-acylhydrazone compounds that are Nav 1.7 and/or Nav 1.8 inhibitors, the processes for the preparation thereof, compositions containing these, uses, kits and treatment methods for treating or preventing pain-related pathologies. The present invention is applicable in the fields of medicinal chemistry, organic synthesis, as well as in the treatment of pain-related disorders.

### BACKGROUND OF INVENTION

Physiological pain is an important protective mechanism developed to alert the body to actual or potential injuries that may endanger its integrity. In general terms, physiological pain can be classified into nociceptive pain and inflammatory pain. Nociceptive pain is characterised by presenting a high activation threshold, which remains until the stimulus that generated it is eliminated. Inflammatory pain, which arises as a response to tissue damage, is characterised by a low activation threshold and is a consequence of the activity of cellular and molecular mediators of the inflammatory process in sensitizing nociceptors (Schaible. Langenbecks Arch. Surg. 2004, 389, 237). When these nociceptive processes remain in the absence of harmful stimuli or in response to non-harmful stimuli, the protective and repair role of pain loses its functionality, constituting a maladaptive framework of neural plasticity and, as a consequence, a pathological state of chronic pain. Among the syndromes included in this classification, neuropathic pain has a high prevalence and impact today (Smith. Pain. 2020, 161, 1: S127; Cavalli. Int. J. Immunopathol. Pharmacol. 2019, 33:2058738419838383; Bouhassira. Rev Neurol (Paris). 2019, 175 (1-2): 16; Scholz. Nature Neurosci., 2002, 5, 1062; Costigan. Annu. Rev. Neurosci., 2009, 32, 1).

Neuropathic pain is defined by the International Association for the Study of Pain (IASP) as pain initiated or caused by a primary dysfunction or injury in the central and/or peripheral nervous system (Dworkin. Clin. J. Pain, 2002, 18 (6), 343). Central neuropathic pain comes from spinal cord injuries or diseases of the central nervous system such as multiple sclerosis or Parkinson's disease (Ducreux. Brain, 2006, 129, 963). Peripheral neuropathic pain, on the other hand, can be caused by trauma, metabolic disorders, chemical neurotoxicity, infection or tumor invasion, among others. Among the most common neuropathic pain syndromes are chemotherapy-induced neuropathic pain, complex regional pain, neuropathy related to viral infection, neuropathy secondary to tumor infiltration, diabetic neuropathy, phantom limb pain, postherpetic neuralgia, trigeminal neuralgia and postsurgical neuralgia (Pak. Curr. Pain Headache Rep., 2018, 22(2), 9).

Currently, there is no specific treatment for the control of pathologies related to neuropathic pain. However, the first-line alternative consists of the use of opioid analgesics, and—as adjuvants—local anesthetics, anticonvulsants and antidepressants. Nevertheless, adverse effects and poor efficacy drastically limit the use of these agents in the control of various pain-related pathologies (Kushnarev. Expert Opin. Investig. Drugs, 2020, 29 (3), 259; Emery. Expert Opin. Ther. Targets, 2016, 20 (8), 975).

Voltage-gated sodium channels (Nav) play a key role in the transmission of pain-related stimuli. These channels are activated in response to membrane depolarization allowing the generation and propagation of action potentials in neurons (and other electrically excitable cells) by controlling the flow of sodium ions across membranes. Structurally, voltage-gated sodium channels are heteromeric transmembrane proteins consisting of an a subunit and two helper ß subunits. The a subunit is organized into four homologous domains (I-IV) each with six transmembrane segments (S1-S6). The S4 segment of each domain is characterised by a conserved region of arginine residues, which act as sensors of the intra- and extracellular electrical environment of the neuron. This mechanism allows transforming alterations of the cellular electric field into specific conformational changes that, in turn, regulate the activation, deactivation and inactivation of voltage-gated sodium channels (Catterall. Nat. Chem. Biol., 2020, 16, 1314; Wisedchaisri. Cell., 2019, 178 (4), 993; Clairfeuille. Science, 2019, 363, 1302).

In mammals, nine α subunits (Nav 1.1-Nav 1.9) and four β helper subunits (β1-β4) have been identified. The a subunits can also be classified as to their susceptibility to tetrodotoxin blockade (TTX), being classified as sensitive to tetrodotoxin (Nav 1.1, Nav 1.2, Nav 1.3, Nav 1.4, Nav 1.6 and Nav 1.7) or resistant to tetrodotoxin (Nav 1.5, Nav 1.8 and Nav 1.9) (Lera-Ruiz. J. Med. Chem., 2015, 58 (18), 7093; Bagal. J. Med. Chem., 2013, 56 (3), 593). Each of these a subunits exhibit a differentiated expression and function profile such that some of them are essential for the proper functioning of organs such as the heart and/or brain. Thus, the non-selective blockage of these channels is related to several types of adverse effects, such as migraine, epilepsy, paralysis and muscle and cardiac syndromes, among others (Bagal. J. Med. Chem., 2013, 56 (3), 593; Bagal. Channels, 2015, 9 (6), 360).

In general terms, sodium channels are distributed mainly in the central and peripheral nervous system, in neurons and glia. Nav channels 1.1, 1.2 and 1.3 are mainly expressed in the brain. Nav 1.4 and Nav 1.5 channels are mainly found in skeletal and cardiac muscles, respectively. Nav 1.6 channels are expressed in the central and peripheral nervous system, while Nav 1.9 channels are selectively expressed in C-type nociceptive fibers in the dorsal root ganglion. On the other hand, Nav 1.7 and Nav 1.8 channels are mainly found in the peripheral nervous system and are directly related to pain transmission processes (Law. Drug Discovery Today, 2019, 24 (7), 1389; Bagal. Channels (Austin), 2015, 9 (6), 360; Lera-Ruiz. J. Med. Chem., 2015, 58 (18), 7093).

Nav 1.7 sodium channels are expressed broadly in the olfactory epithelium, sympathetic ganglion, and dorsal root ganglion, predominantly in the C and Aδ nociceptivefibers. A large amount of evidence supports the important role of Nav 1.7 sodium channels in pain transmission processes. For example, gain-of-function-related mutations in the gene (SCN9A), which encodes the Nav 1.7 sodium channel, are associated with extreme pain disorders such as congenital pain hypersensitivity, paroxysmal extreme pain disorder, and primary erythromelalgia. On the other hand, mutations related to loss of gene function (SCN9A) are related to congenital insensitivity to pain in individuals who, in general terms, are free of motor or cognitive impairment (Vetter. Pharmacology & Therapeutics, 2017, 172, 73; Ahuja. Science, 2015, 350 (6267), 1491; Kingwell. Nat. Rev. Drug Discov., 2019, 18, 321; Safina. J. Med. Chem., 2021, 64, 2953; Luo. J. Med. Chem., 2019, 62, 831; Bankar. Cell Reports, 2018, 24, 3133).

Nav 1.8 sodium channels show increased expression in the peripheral nervous system, largely (but not exclusively) in C-type nociceptive fibers in the dorsal root ganglion. Recent evidence that includes elevated Nav 1.8 expression levels in chronic pain states, Nav 1.8 knockout animal data, and analgesic activity of desensitizing oligodeoxynucleotides specific for Nav 1.8, among others (Brown. Bioorg. Med. Chem., 2019, 27 (1), 230; Payne. Br. J. Pharmacol., 2015, 172 (10), 2654; Bagal. Med. Chem. Lett. 2015, 6 (6) 650; Kort. J. Med. Chem. 2008, 51, 407; Zhang. Neuropharmacology, 2010, 59, 201 and 207), support the role of the Nav 1.8 sodium channel in the development and process of pain-related pathologies (Kingwell. Nat. Rev. Drug Discov., 2019, 18, 321; Law. Drug Discovery Today, 2019, 24 (7), 1389; Bagal. Channels (Austin), 2015, 9 (6), 360; Lera-Ruiz. J. Med. Chem., 2015, 58 (18), 7093).

In this way, the voltage-gated sodium channels Nav 1.7 and Nav 1.8 are considered promising therapeutic targets for the treatment of neuropathic pain-related dysfunctions (Kornecook. J. Pharmacol. Exp. Ther., 2017, 362, 146; Kingwell. Nat. Rev. Drug Discov., 2019, 18, 321; Bagal. Channels (Austin), 2015, 9 (6), 360; Lera-Ruiz. J. Med. Chem., 2015, 58 (18), 7093; Deuis. Neuropharmacology, 2017, 127, 87 and 108; Kushnarev. Expert Opin. Investig. Drugs, 2020, 29 (3), 259; McKerrall. Bioorganic & Medicinal Chemistry Lett., 2018, 28, 3141; Emery. Expert Opin. Ther. Targets, 2016, 20 (8), 975; Bagal. Bioorganic & Medicinal Chemistry Lett., 2014, 24, 3690).

A large number of compounds have been described in the literature for their ability to act as blockers of Nav 1.7 and 1.8 sodium channels. However, they present a great structural diversity, a fact that does not allow a common pharmacophoric group to be established.

The patent literature contains several examples of compounds that act as sodium channel blockers. In particular, blockers of the selective sodium channels Nav 1.7 are described in U.S. Pat. Nos. 10,550,080, 9,765,029 and 10,000,475. Additionally, some documents describe selective blockers of Nav 1.8 sodium channels such as WO2020261114, WO2020092667, U.S. Pat. No. 9,163,042, WO2014120808, WO2014120815, WO2018213426, WO2019014352, WO2015006280 and U.S. Pat. No. 7,928,107. These documents disclose compounds having distinct structures of the present invention.

Still, there are patent documents describing dual inhibitors of Nav 1.7 and 1.8, including WO2018235851, U.S. Pat. No. 8,629,149, JP2017001991 that disclose, respectively, pyridyl amines, oxopiperazine derivatives and benzoxazolones. However, all of these prior art documents disclose compounds with structures and physicochemical characteristics distinct from the present invention.

In this context, it is advantageous to develop new alternatives of compounds that can act as inhibitors of Nav 1.7 and/or Nav 1.8 that have adequate pharmacological action and, preferably, provide mitigated adverse effects. Therefore, the present invention relates to N-acylhydrazone derivatives endowed with novel and inventive step as an alternative and/or complement to the treatment of pain-related diseases.

### SUMMARY OF INVENTION

The present invention discloses N-acylhydrazone compounds with inhibitory activity of Nav 1.7 and/or 1.8, against pain-related pathologies, in addition to compositions, uses, kits, treatment methods and related preparation processes.

The present invention relates to compound(s) of Formula (I):

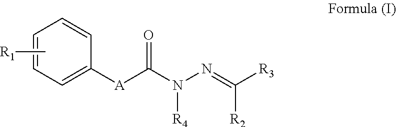

or a pharmaceutically acceptable salt, hydrate, solvate, and isomer thereof, wherein:
A is selected from the group consisting of

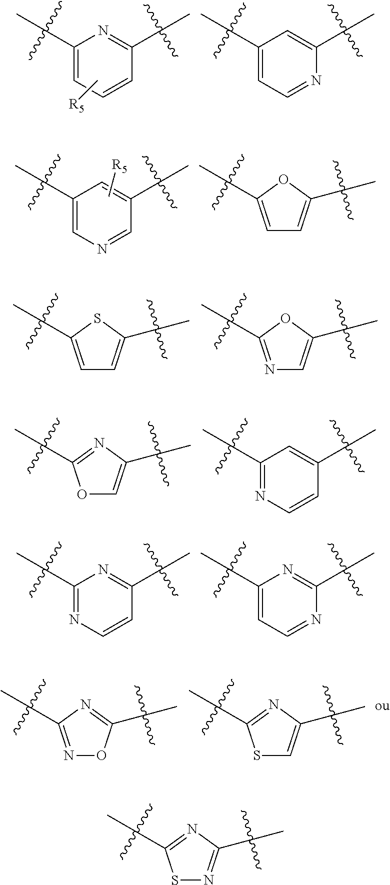

R1 is selected from the group consisting of hydrogen, halogen, linear or branchedC1-6 alkoxy, 2-morpholinoethoxy, C3-6 cycloalkyloxy, or C1-5 haloalkyloxy;
R2 and R4 are independently selected from the group consisting of hydrogen or branched or linear C1-6 alkyl;
R3 is selected from the group consisting of hydrogen, heterocycle or substituted heterocycle, or R6;
R5 is selected from the group consisting of hydrogen, halogen, trifluoromethyl.
R6 is

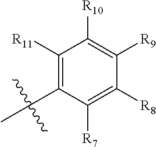

R7, R8, R9, R10 and R11 are independently selected from the group consisting of hydrogen, halogen, linear or branched C1-6 alkoxy.

Additionally, the present invention also relates to compositions comprising one or more compound(s) of Formula (I) or pharmaceutically acceptable salts, solvates, and isomers thereof; and one or more pharmaceutically acceptable excipients.

Additionally, kits according to the present invention may comprise such compositions and application devices, which may include ampoules, syringes, and others. Alternatively, kits according to the present invention comprise more than one compound of Formula (I) arranged in one or more dosage forms, including without limitation, tablets, accompanied by instructions for administration.

The present invention further relates to methods of treating, preventing, alleviating, suppressing and/or controlling diseases related to neuropathic pain. Also taught are uses of compound(s) of general Formula (I) for preparing a medicament for the treatment of neuropathic pain-related pathologies. Finally, the present invention teaches processes of obtaining compound(s) of general Formula (I).

### DETAILED DESCRIPTION OF INVENTION

The present invention features in a first embodiment, the compound of Formula (I):

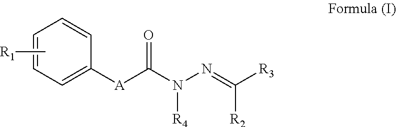

or a pharmaceutically acceptable salt, hydrate, solvate, and isomer thereof, wherein:

A is selected from the group consisting of

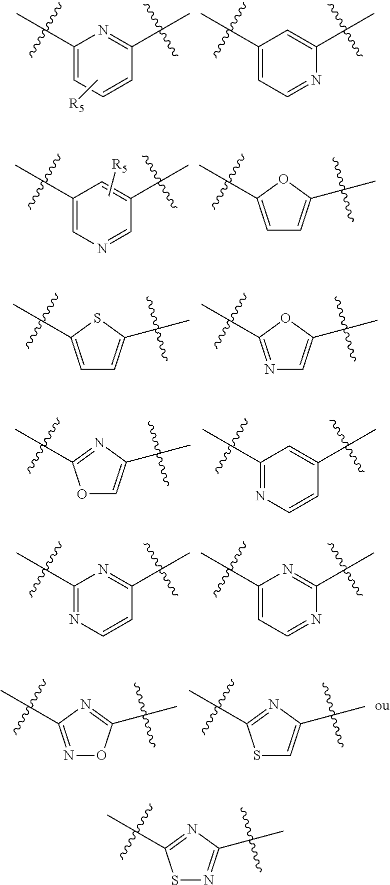

R1 is selected from the group consisting of hydrogen, halogen, linear or branchedC1-6 alkoxy, 2-morpholinoethoxy, C3-6 cycloalkyloxy, or C1-5 haloalkyloxy;
R2 and R4 are independently selected from the group consisting of hydrogen or branched or linear C1-6 alkyl;
R3 is selected from the group consisting of hydrogen, heterocycle or substituted heterocycle, or R6;
R5 is selected from the group consisting of hydrogen, halogen, trifluoromethyl.
R6 is

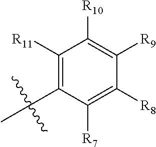

R7, R8, R9, R10 and R11 are independently selected from the group consisting of hydrogen, halogen, linear or branchedC1-6 alkoxy.

In one embodiment, A is

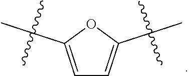 [SMILES: *c1ccc(*)o1]

In another preferred embodiment, A is

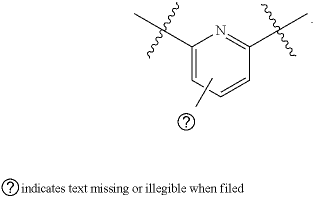

In a further preferred embodiment, A is

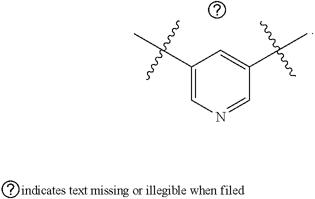 [SMILES: "." ; *c1cncc(*)c1]

In another preferred embodiment, A is

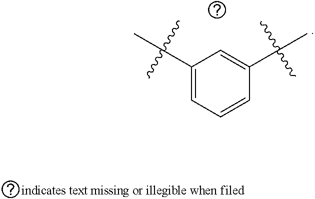 [SMILES: "." ; *c1cccc(*)c1]

In another preferred embodiment, A is selected from

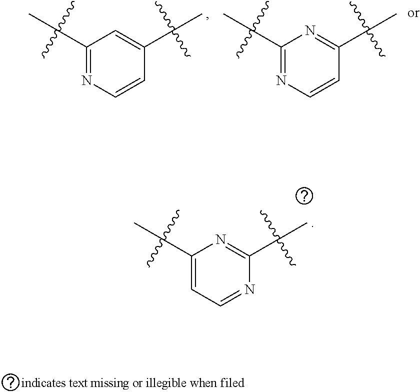 [SMILES: "or" ; "," ; *c1ccnc(*)c1 ; *c1ccnc(*)n1 ; "." ; *c1ccnc(*)n1]

In a further preferred embodiment, A is selected from the group consisting of

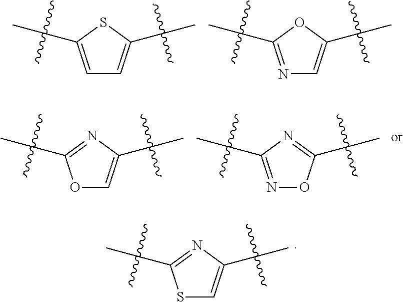

In one embodiment, R1 is selected from the group consisting of hydrogen, chloro, bromo, fluoro, methoxy, ethoxy, isopropoxy, propoxy, butoxy, 2-morpholinoethoxy, cyclopropoxy; cyclopentyloxy, cyclohexyloxy, cyclobutyloxy, or trifluoromethoxy. In a preferred embodiment, R1 is selected from the group consisting of hydrogen, chloro, methoxy, ethoxy, isopropoxy, propoxy, butoxy, 2-morpholinoethoxy, cyclopentyloxy, or trifluoromethoxy. In a further preferred embodiment, R1 is selected from the group consisting of chloro, methoxy, ethoxy, isopropoxy or 2-morpholinoethoxy. In a further preferred embodiment, R1 is selected from the group consisting of chloro, methoxy, ethoxy, isopropoxy, propoxy, butoxy, trifluoromethoxy or cyclopentyloxy. In a further preferred embodiment, R1 is methoxy.

In one embodiment, R2 and R4 are independently selected from the group consisting of hydrogen, methyl, ethyl, propyl, or isopropyl. In a preferred embodiment, R2 and R4 are independently selected from hydrogen or methyl. In a further preferred embodiment, R2 and R4 are each hydrogen. In a further preferred embodiment, R4 is selected from hydrogen or methyl.

In one embodiment, R3 is selected from the group consisting of hydrogen, thiophene, furan, 1H-pyrrol-2-yl, 1-methyl-1H-pyrrol-2-yl, 1-ethyl-1H-pyrrol-2-yl, 1H-imidazol-5-yl, 1H-pyrazol-5-yl, 2-oxazole, 2-thiazole, 5-oxazole, 5-thiazole, 1,3,4-oxadiazole, 1,3,4-thiadiazole, 3-isoxazole, 5-isoxazole, 3-isothiazole, 5-isothiazole, isoxazolpyridin-3-yl; pyridin-4-yl, pyridin-2-yl; 2-morpholinopyridin-3-yl, 4-((dimethylamino)methyl)thiophen-2-yl, or R6. In a preferred embodiment, R3 is selected from the group consisting of hydrogen, thiophene, pyridin-3-yl; pyridin-4-yl, pyridin-2-yl; 2-morpholinopyridin-3-yl, 4-((dimethylamino)methyl) thiophen-2-yl, or R6. In a further preferred embodiment, R3 is selected from the group consisting of thiophene, 4-((dimethylamino)methyl) thiophen-2-yl, pyridinyl, 2-morpholinopyridin-3-yl or R6. In a further preferred embodiment, R3 is selected from the group consisting of thiophene, 2-morpholinopyridin-3-yl or R6. In another preferred embodiment, R3 is thiophene or R6. In another preferred embodiment, R3 is R6.

In one embodiment, R5 is selected from the group consisting of hydrogen, chloro, bromo, fluoro, or trifluoromethyl. In a preferred embodiment, R5 is selected from the group consisting of hydrogen, fluoro or trifluoromethyl. In a further preferred embodiment, R5 is selected from hydrogen or fluorine.

In one embodiment R7, R8, R9, R10 and R11 are independently selected from the group consisting of hydrogen, chlorine, fluorine, bromine, methoxy, ethoxy, propoxy or isoproxy.

In a preferred embodiment, R7 is selected from hydrogen, chlorine, fluorine or methoxy. In a further preferred embodiment, R7 is hydrogen.

In a preferred embodiment, R8 and R10 are independently selected from hydrogen or methoxy.

In a preferred embodiment, R9 is hydrogen.

In a preferred embodiment, R11 is selected from the group consisting of hydrogen, chlorine or fluorine. In a further preferred embodiment, R11 is hydrogen.

In a preferred embodiment, the compound(s) of Formula (I) is selected from the group consisting of:
(E)-5-(4-chlorophenyl)-N′-(thiophen-2-ylmethylene) furan-2-carbohydrazide (Compound 1);
(E)-5-(4-chlorophenyl)-N′-(3,5-dimethoxybenzylidene) furan-2-carbohydrazide (Compound 2);
(E)-5-(4-ethoxyphenyl)-N′-(thiophen-2-ylmethylene) furan-2-carbohydrazide (Compound 3);
(E)-5-(4-ethoxyphenyl)-N′-((2-morpholinopyridin-3-yl)methylene) furan-2-carbohydrazide (Compound 4);
(E)-N′-((4-((dimethylamino)methyl) thiophen-2-yl)methylene)-5-(4-ethoxyphenyl) furan-2-carbohydrazide (Compound 5);
(E)-5-(4-ethoxyphenyl)-N′-(pyridin-3-ylmethylene) furan-2-carbohydrazide (Compound 6);
(E)-5-(4-isopropoxyphenyl)-N′-(thiophen-2-ylmethylene) furan-2-carbohydrazide (Compound 7);
(E)-N′-((4-((dimethylamino)methyl) thiophen-2-yl)methylene)-5-(4-isopropoxyphenyl) furan-2-carbohydrazide (Compound 8);
(E)-5-(4-(2-morpholinoethoxy)phenyl)-N′-(thiophen-2-ylmethylene) furan-2-carbohydrazide (Compound 9);
(E)-6-(4-chlorophenyl)-N′-(3,5-dimethoxybenzylidene) picolinohydrazide (Compound 10);
(E)-6-(4-chlorophenyl)-N′-(thiophen-2-ylmethylene) picolinohydrazide (Compound 11);
(E)-6-(4-ethoxyphenyl)-N′-(thiophen-2-ylmethylene) picolinohydrazide (Compound 12);
(E)-6-(4-isopropoxyphenyl)-N′-(thiophen-2-ylmethylene) picolinohydrazide (Compound 13);
(E)-N′-(3,5-dimethoxybenzylidene)-6-(4-methoxyphenyl) picolinohydrazide (Compound 14);
(E)-N′-(3,5-dimethoxybenzylidene)-6-(4-propoxyphenyl) picolinohydrazide (Compound 15);
(E)-5-(4-chlorophenyl)-N′-(3,5-dimethoxybenzylidene) nicotinohydrazide (Compound 16);
(E)-N′-(3,5-dimethoxybenzylidene)-5-(4-(trifluoromethoxy)phenyl) nicotinohydrazide (Compound 17);
(E)-N′-(3,5-dimethoxybenzylidene)-5-(4-ethoxyphenyl) nicotinohydrazide (Compound 18);
(E)-N′-(3,5-dimethoxybenzylidene)-5-(4-isopropoxyphenyl) nicotinohydrazide (Compound 19);
(E)-2-(4-chlorophenyl)-N′-(3,5-dimethoxybenzylidene) isonicotinohydrazide (Compound 20);
(E)-N′-(3,5-dimethoxybenzylidene)-2-(4-(trifluoromethoxy)phenyl) isonicotinohydrazide (Compound 21);
(E)-N′-(3,5-dimethoxybenzylidene)-2-(4-ethoxyphenyl) isonicotinohydrazide (Compound 22);
(E)-N′-(3,5-dimethoxybenzylidene)-2-(4-isopropoxyphenyl) isonicotinohydrazide (Compound 23);
(E)-N′-(3,5-dimethoxybenzylidene)-6-(4-isopropoxyphenyl) picolinohydrazide (Compound 24);
(E)-N′-(3,5-dimethoxybenzylidene)-5-(4-methoxyphenyl) nicotinohydrazide (Compound 25);
(E)-N′-(3,5-dimethoxybenzylidene)-5-(4-propoxyphenyl) nicotinohydrazide (Compound 26);
(E)-5-(4-butoxyphenyl)-N′-(3,5-dimethoxybenzylidene) nicotinohydrazide (Compound 27);
(E)-5-(4-(cyclopentyloxy)phenyl)-N′-(3,5-dimethoxybenzylidene) nicotinohydrazide (Compound 28);
(E)-N′-(3,5-dimethoxybenzylidene)-2-(4-methoxyphenyl) isonicotinohydrazide (Compound 29);
(E)-N′-(3,5-dimethoxybenzylidene)-2-(4-propoxyphenyl) isonicotinohydrazide (Compound 30);
(E)-N′-(3,5-dimethoxybenzylidene)-6-(4-ethoxyphenyl) picolinohydrazide (Compound 31);
(E)-6-(4-ethoxyphenyl)-N′-(2-fluorobenzylidene) picolinohydrazide (Compound 32);
(E)-N′-(2-chlorobenzylidene)-6-(4-ethoxyphenyl) picolinohydrazide (Compound 33);
(E)-N′-(2,6-dichlorobenzylidene)-6-(4-ethoxyphenyl) picolinohydrazide (Compound 34);
(E)-N′-(2-chloro-6-fluorobenzylidene)-6-(4-ethoxyphenyl) picolinohydrazide (Compound 35);
(E)-6-(4-ethoxyphenyl)-N′-(2-methoxybenzylidene) picolinohydrazide (Compound 36);
(E)-6-(4-ethoxyphenyl)-N′-((2-morpholinopyridin-3-yl)methylene) picolinohydrazide (Compound 37);
(E)-2-(4-butoxyphenyl)-N′-(3,5-dimethoxybenzylidene) isonicotinohydrazide (Compound 38);
(E)-2-(4-(cyclopentyloxy)phenyl)-N′-(3,5-dimethoxybenzylidene) isonicotinohydrazide (Compound 39);
(E)-N′-(3,5-dimethoxybenzylidene)-6-(4-(trifluoromethoxy)phenyl) picolinohydrazide (Compound 40);
(E)-6-(4-butoxyphenyl)-N′-(3,5-dimethoxybenzylidene) picolinohydrazide (Compound 41);
(E)-4-(4-chlorophenyl)-N′-(3,5-dimethoxybenzylidene) picolinohydrazide (Compound 42);
(E)-4-(4-chlorophenyl)-N′-(thiophen-2-ylmethylene) picolinohydrazide (Compound 43);
(E)-N′-(3,5-dimethoxybenzylidene)-4-(4-ethoxyphenyl) picolinohydrazide (Compound 44);
(E)-4-(4-ethoxyphenyl)-N′-(thiophen-2-ylmethylene) picolinohydrazide (Compound 45);
(E)-N′-(3,5-dimethoxybenzylidene)-4-(4-isopropoxyphenyl) picolinohydrazide (Compound 46);
(E)-4-(4-isopropoxyphenyl)-N′-(thiophen-2-ylmethylene) picolinohydrazide (Compound 47);
(E)-4-(4-(cyclopentyloxy)phenyl)-N′-(3,5-dimethoxybenzylidene) picolinohydrazide (Compound 48);
(E)-N′-(3,5-dimethoxybenzylidene)-4-(4-(trifluoromethoxy)phenyl) picolinohydrazide (Compound 49);
(E)-4-(4-(cyclopentyloxy)phenyl)-N′-(thiophen-2-ylmethylene) picolinohydrazide (Compound 50);
(E)-N′-(thiophen-2-ylmethylene)-4-(4-(trifluoromethoxy)phenyl) picolinohydrazide (Compound 51);
(E)-2-(4-chlorophenyl)-N′-(3,5-dimethoxybenzylidene)pyrimidine-4-carbohydrazide (Compound 52);
(E)-N′-(3,5-dimethoxybenzylidene)-2-(4-ethoxyphenyl)pyrimidine-4-carbohydrazide (Compound 53);
(E)-N′-(3,5-dimethoxybenzylidene)-2-(4-isopropoxyphenyl)pyrimidine-4-carbohydrazide (Compound 54);
(E)-2-(4-(cyclopentyloxy)phenyl)-N′-(3,5-dimethoxybenzylidene)pyrimidine-4-carbohydrazide (Compound 55);
(E)-N′-(3,5-dimethoxybenzylidene)-2-(4-(trifluoromethoxy)phenyl) pyrimidine-4-carbohydrazide (Compound 56);
(E)-N′-(3,5-dimethoxybenzylidene)-2-(4-methoxyphenyl)pyrimidine-4-carbohydrazide (Compound 57);
(E)-N′-(3,5-dimethoxybenzylidene)-2-(4-propoxyphenyl)pyrimidine-4-carbohydrazide (Compound 58);
(E)-2-(4-butoxyphenyl)-N′-(3,5-dimethoxybenzylidene)pyrimidine-4-carbohydrazide (Compound 59);
(E)-4-(4-chlorophenyl)-N′-(3,5-dimethoxybenzylidene)pyrimidine-2-carbohydrazide (Compound 60);
(E)-N′-(3,5-dimethoxybenzylidene)-4-(4-ethoxyphenyl)pyrimidine-2-carbohydrazide (Compound 61);
(E)-N′-(3,5-dimethoxybenzylidene)-4-(4-isopropoxyphenyl)pyrimidine-2-carbohydrazide (Compound 62);
(E)-4-(4-(cyclopentyloxy)phenyl)-N′-(3,5-dimethoxybenzylidene)pyrimidine-2-carbohydrazide (Compound 63);
(E)-N′-(3,5-dimethoxybenzylidene)-4-(4-(trifluoromethoxy)phenyl) pyrimidine-2-carbohydrazide (Compound 64);
(E)-N′-(3,5-dimethoxybenzylidene)-4-(4-methoxyphenyl)pyrimidine-2-carbohydrazide (Compound 65);
(E)-N′-(3,5-dimethoxybenzylidene)-4-(4-propoxyphenyl)pyrimidine-2-carbohydrazide (Compound 66);
(E)-4-(4-butoxyphenyl)-N′-(3,5-dimethoxybenzylidene)pyrimidine-2-carbohydrazide (Compound 67);
(E)-N′-(3,5-dimethoxybenzylidene)-6-(4-ethoxyphenyl)-4-fluoropicolinohydrazide (Compound 68);
(E)-N′-(3,5-dimethoxybenzylidene)-6-(4-ethoxyphenyl)-4-trifluoromethyl) picolinohydrazide (Compound 69);
(E)-N′-(3,5-dimethoxybenzylidene)-5-(4-ethoxyphenyl)-4-fluoronicotinohydrazide (Compound 70);
(E)-N′-(3,5-dimethoxybenzylidene)-5-(4-methoxyphenyl) furan-2-carbohydrazide (Compound 71);
(E)-N′-(3,5-dimethoxybenzylidene)-5-(4-methoxyphenyl)thiophene-2-carbohydrazide (Compound 72);
(E)-N′-(3,5-dimethoxybenzylidene)-2-(4-methoxyphenyl) oxazole-5-carbohydrazide (Compound 73);
(E)-N′-(3,5-dimethoxybenzylidene)-2-(4-methoxyphenol) oxazole-4-carbohydrazide (Compound 74);
(E)-N′-(3,5-dimethoxybenzylidene)-3-(4-methoxyphenol)-1,2,4-oxadiazole-5-carbohydrazide (Compound 75);
(E)-N′-(3,5-dimethoxybenzylidene)-2-(4-methoxyphenyl) thiazole-4-carbohydrazide (Compound 76);
(E)-N′-(3,5-dimethoxybenzylidene)-5-(4-methoxyphenyl)-1,2,4-thiadiazole-3-carbohydrazide (Compound 77);
(E)-5-(4-chlorophenyl)-N′-(3,5-dimethoxybenzylidene)-N′-methylfuran-2-carbohydrazide (Compound 78);
(E)-N′-(3,5-dimethoxybenzylidene)-6-(4-ethoxyphenyl)-N′-methylpicolinohydrazide (Compound 79).

In one embodiment, the Formula (I) compound(s) are selective inhibitors of the voltage-gated sodium channels Nav 1.7 and/or Nav 1.8. In a preferred embodiment, the compound(s) Formula (I) are dual inhibitors of the voltage-gated sodium channels Nav 1.7 and Nav 1.8.

In some embodiments, the compound(s) of Formula (I) may be basic in nature, and accordingly pharmaceutically acceptable salts may be obtained by the addition of organic or inorganic acids. Non-limiting examples of organic acids that may be used are fumaric, maleic, benzoic, lactic acids, among others. Among the inorganic acids may be mentioned hydrochloric acid, phosphoric acid, hydrobromic acid, sulfuric acid, nitric acid, among others.

In some embodiments, the compound(s) of Formula (I) may be obtained in the form of crystals, which may optionally be presented as pharmaceutically acceptable solvates, wherein the solvent is incorporated in stoichiometric proportions or not into the crystal lattice. In further embodiments, the crystallization solvent is water, resulting in pharmaceutically acceptable hydrates.

Finally, in some embodiments, the compound(s) of Formula (I) may exhibit more than one isomer, including without limitation, spatial isomerism such as geometric and optical isomerism.

### Definitions

For the purpose of clarity or elucidation of the terms used in the present invention, the following definitions are presented, and the scope is not limited thereto.

The term “straight or branched C1-6 alkyl” refers to saturated straight or branched chain hydrocarbons such as methyl, ethyl, propyl, butyl, isopropyl, tert-butyl, pentyl, hexyl, not limited thereto.

The term “linear or branchedC1-6 alkoxy” refers to alkyl groups bonded to a radical oxygen, including methoxy, ethoxy, propoxy, butoxy, pentoxy, hexoxy, isoproxy, isobutoxy, tert-butoxy, and is not limited thereto.

The term “C1-6haloalkyloxy” refers to alkoxy groups bonded to a halogen, including groups such as trifluoromethoxy, difluoromethoxy, fluoromethoxy, chloromethoxy, dichloromethoxy, trichloromethoxy, 2,2,2-trifluoroethoxy, 2,2-difluoroethoxy, 2-fluoroethoxy, 2-chloroethoxy, 2,2-dichloroethoxy, 2,2,2-trichloroethoxy, 3,3,3-trifluoropropoxy, 3,3-difluoropropoxy, 3-fluoroproxy, 3-chloropropoxy, 3,3-dichloropropoxy, 3,3,3-trichloropropoxy, 4,4,4-trifluorobutoxy, 5,5,5-trifluoropentoxy, not limited thereto.

The term “C3-6 cycloalkyloxy” refers to groups such as cyclopropoxy; cyclopentyloxy; cyclohexyloxy; cyclobutyloxy, not limited thereto.

The term “heterocycle” or “substituted heterocycle” refers to groups such as thiophene, furan, 1H-pyrrol-2-yl, 1-methyl-1H-pyrrol-2-yl, 1-ethyl-1H-pyrrol-2-yl, 1H-imidazol-5-yl, 1H-pyrazol-5-yl, 2-oxazole, 2-thiazole, 5-oxazole, 5-thiazole, 1,3,4-oxadiazole, 1,3,4-thiadiazole, 3-isoxazole, 5-isoxazole, 3-isothiazole, 5-isothiazole, isoxazolpyridin-3-yl; pyridin-4-yl, pyridin-2-yl; 2-morpholinopyridin-3-yl, 4-((dimethylamino)methyl) thiophen-2-yl, not limited thereto.

In a second embodiment, the present invention features a composition comprising a therapeutically effective amount of compound(s) of Formula (I) of the present invention or a pharmaceutically acceptable salt, hydrate, solvate, and isomerthereof; and one or more pharmaceutically acceptable excipients.

Pharmaceutically acceptable excipients are considered to be any substance, other than the active pharmaceutical ingredient, that has been evaluated for safety and that is intentionally added to the dosage form. Such excipients are selected according to the pharmaceutical dosage form of interest, its route of administration, physicochemical compatibility with the active ingredient, and the effect on efficacy.

Furthermore, said excipients are widely known in the state of art and are classified according to their function, including without limitation diluents, binders, disintegrants or disaggregants, lubricants, suspending agents, thickeners, solvents, surfactants, glidants, anti-caking or flowing agents, coating agents, plasticizers, sweeteners, sweeteners, isotonicity agents, dyes and pigments, preservatives, antioxidants, pH modifying or control agents, complexing agents, chelating agents, flavoring, flavoring, viscosity modifying agents, opacifiers, permeation promoters, among others.

The pharmaceutical compositions may be administered by various routes including, but not limited to, oral, sublingual, nasal, parenteral, injectable, submuscular, topical, transdermal, ocular, rectal.

In a third embodiment, the present invention features the use of compound(s) of Formula (I) or a pharmaceutically acceptable salt, hydrate, solvate, and isomer thereof to prepare a medicament for treating neuropathic pain-related pathologies.

In a preferred embodiment, said pathologies are selected from the group consisting of peripheral neuropathic pain, chemotherapy-induced neuropathy, complex regional pain, neuropathy related to viral infection, neuropathy secondary to tumor infiltration, diabetic neuropathy, phantom limb pain, postherpetic neuralgia, trigeminal neuralgia and postsurgical neuralgia.

In a fourth embodiment, the present invention features a method of treating, preventing, alleviating, suppressing, and/or controlling neuropathic pain-related pathologies comprising administering an effective amount of compound(s) of Formula (I) or a pharmaceutically acceptable salt, hydrate, solvate, and isomer thereof. In a preferred embodiment, the method is for the treatment, prevention, alleviation, suppression and/or control of peripheral neuropathic pain, chemotherapy-induced neuropathy, complex regional pain, neuropathy related to viral infection, neuropathy secondary to tumor infiltration, diabetic neuropathy, phantom limb pain, postherpetic neuralgia, trigeminal neuralgia and postsurgical neuralgia. In a further preferred embodiment, the administration of the at least one compound of Formula (I) is selected from the group comprising oral, sublingual, nasal, parenteral, injectable, submuscular, topical, transdermal, ocular, and rectal.

In a fifth embodiment of the invention, there are provided processes of obtaining compound(s) of Formula (I) comprising the following steps:
(a) Forming the intermediate of Formula III:

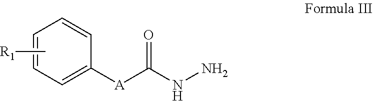

from the hydrazinolysis reaction of an intermediate of Formula IV:

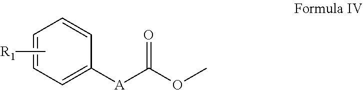

(b) obtaining a Formula I compound wherein R4 is hydrogen;

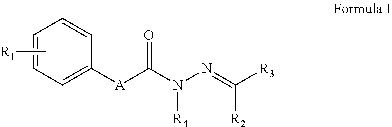

from condensation of intermediates of Formula II:

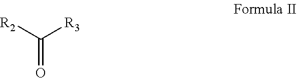

and Formula III, with or without presence of catalyst and a suitable solvent;
wherein, A, R1, R2, R3, R5, R6, R7, R8, R9, R10, R11 are independently selected from the groups previously described.

In one embodiment, the process further comprises a step (c) of forming compound(s) of Formula (I) wherein R4 is linear or branched C1-6 alkyl from the nucleophilic substitution reaction of a compound obtained in (b), with linear or branched C1-6 alkyl halides in the presence of inorganic base and polar aprotic solvents.

In one embodiment of the process, in step (b) said catalyst is selected from concentrated hydrochloric acid, acetic acid, trifluoroacetic acid, formic acid, or combinations thereof and said solvent is selected from dimethylformamide, alcohols, or combinations thereof. In another embodiment, in step (c) said inorganic base may be selectedfrom K2CO3 or NaH.

The compound(s) of Formula (I) of the present invention were prepared from the synthetic route described in General Scheme 1. However, those skilled in the art will readily appreciate that additional detailing and/or modifications to the arrangement of one or more steps may be performed without departing from the processes taught herein. Such variations may be, without limitation, combinations of solvents and catalysts, including stereoselectives, protecting groups, among others.

In the following General Scheme 1, the formation of the intermediates of Formula II, III and IV is described, in addition to the formation of the compound(s) of Formula (I), wherein Formula Ia corresponds to compounds in which R4 is hydrogen and Formula Ib corresponds to compounds in which R4 is a C1-6 alkylgroup (represented by R in the scheme), from these intermediates.

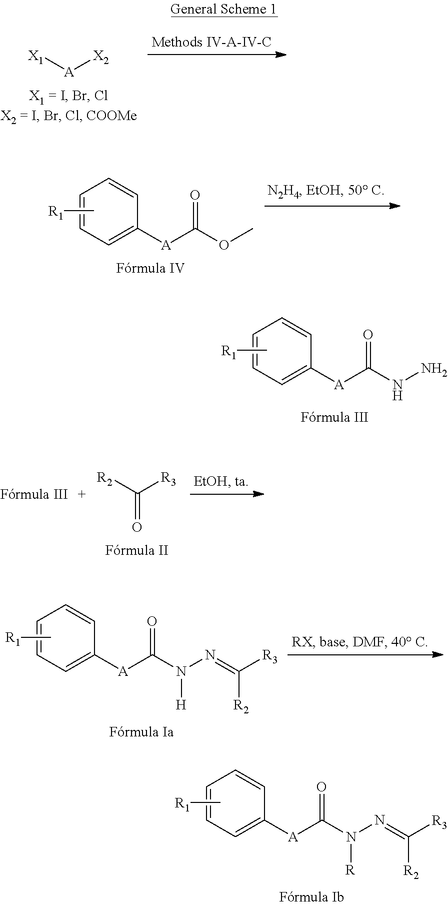

As illustrated in General Scheme 1, compounds of Formula Ia can be prepared from condensation reactions of the intermediates of Formula III with the intermediates of Formula II, without or upon acid catalysis using concentrated hydrochloric acid, acetic acid, trifluoroacetic acid or formic acid in suitable solvents such as DMF or alcohols such as methanol, ethanol or propanol. Compounds of Formula Ib (R═R4 being C1-6 alkyl group), can be obtained from an additional step, starting from compounds of Formula Ia, by nucleophilic substitution reactions with corresponding alkyl halides, in the presence of an inorganic base such as K2CO3 or NaH and polar aprotic solvents. In turn, intermediates of Formula III can be prepared by hydrazinolysis reaction from the corresponding esters (Formula IV) obtained following methods IV-A to IV-C. Intermediates of Formula II can be obtained commercially. The details of such methodologies are described below.

### Obtaining Compounds of Formula IV:

### Method IV-A:

### Intermediates IV-1-IV-33/IV-52-IV-59

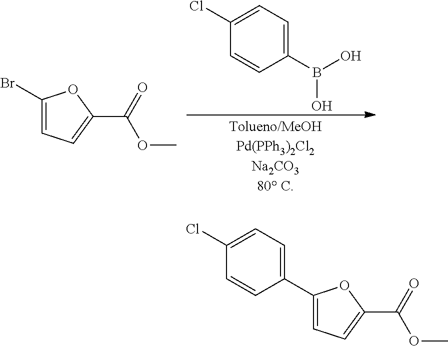

Into a 25-mL round-bottom flask were added 1 mmol of methyl 5-bromofuran-2-carboxylate, 1.5 mmol of 4-chlorophenylboronic acid, and 0.075 mmol of Pd(PPh3) 2Cl2. 5 mL of toluene and 5 mL MeOH were added. Once the reactants dissolved, 20 mmol Na2CO3 was added as a 2M solution in distilled water. Subsequently, the reaction mixture was allowed to stir at 80° C. After total consumption of the starting reagent (monitored by CCF), the reaction mixture was filtered on Celite and concentrated. Subsequently, the resulting oil was dissolved with distilled water (25 mL) and extracted with AcOEt (3×25 mL). The organic phases were pooled, dried over anhydrous magnesium sulfate, filtered and concentrated to give the product, which was purified by column chromatography (n-hexane/AcOEt: 100/0→60/40) (Table 1).

For compounds IV-17 through IV-28, the solvent was replaced with dioxane, the catalyst used was Pd(PPh3) 4, and the temperature was maintained at 110° C. for 12 hours (Table 1).

For compounds IV-29 to IV-33 the solvent was replaced with dioxane and the temperature was maintained at 110° C. for 12 hours (Table 1).

TABLE 1
Intermediates obtained, from the corresponding reagents, following
Method IV-A.

| Inter- | |
|---|---|
| me- | |
| diate | Structure |
| IV-1 | 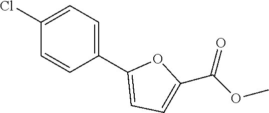 [SMILES: COC(=O)c1ccc(-c2ccc(Cl)cc2)o1] |
| IV-2 | 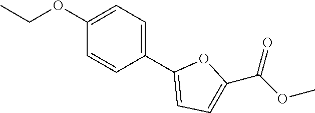 [SMILES: CCOc1ccc(-c2ccc(C(=O)OC)o2)cc1] |
| IV-3 | 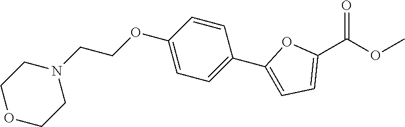 [SMILES: COC(=O)c1ccc(-c2ccc(OCCN3CCOCC3)cc2)o1] |
| IV-4 | 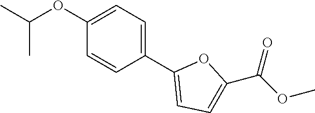 [SMILES: COC(=O)c1ccc(-c2ccc(OC(C)C)cc2)o1] |
| IV-5 | 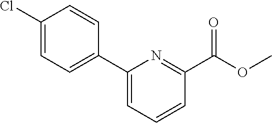 [SMILES: COC(=O)c1cccc(-c2ccc(Cl)cc2)n1] |
| IV-6 | 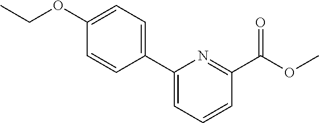 [SMILES: CCOc1ccc(-c2cccc(C(=O)OC)n2)cc1] |
| IV-7 | 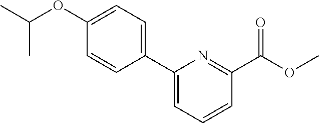 [SMILES: COC(=O)c1cccc(-c2ccc(OC(C)C)cc2)n1] |
| IV-8 | 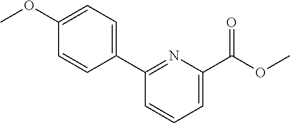 |
| IV-9 | 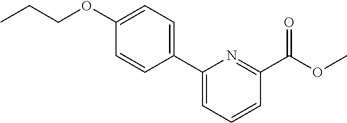 [SMILES: CCCOc1ccc(-c2cccc(C(=O)OC)n2)cc1] |
| IV-10 | 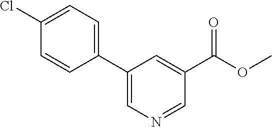 [SMILES: COC(=O)c1cncc(-c2ccc(Cl)cc2)c1] |
| IV-11 | 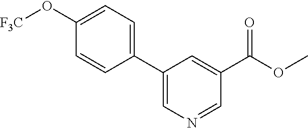 [SMILES: COC(=O)c1cncc(-c2ccc(OC(F)(F)F)cc2)c1] |
| IV-12 | 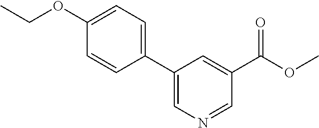 [SMILES: CCOc1ccc(-c2cncc(C(=O)OC)c2)cc1] |
| IV-13 | 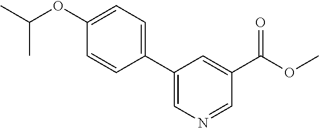 [SMILES: COC(=O)c1cncc(-c2ccc(OC(C)C)cc2)c1] |
| IV-14 | 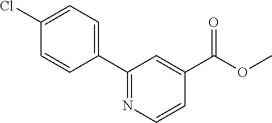 [SMILES: COC(=O)c1ccnc(-c2ccc(Cl)cc2)c1] |
| IV-15 | 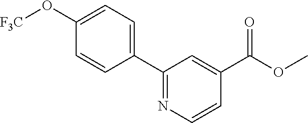 [SMILES: COC(=O)c1ccnc(-c2ccc(OC(F)(F)F)cc2)c1] |
| IV-16 | 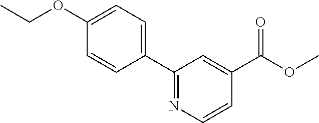 [SMILES: CCOc1ccc(-c2cc(C(=O)OC)ccn2)cc1] |
| IV-17 | 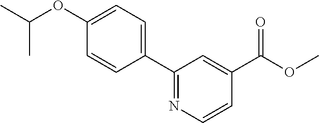 [SMILES: COC(=O)c1ccnc(-c2ccc(OC(C)C)cc2)c1] |
| IV-18 | 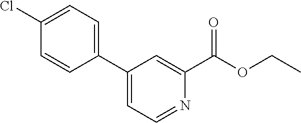 [SMILES: CCOC(=O)c1cc(-c2ccc(Cl)cc2)ccn1] |
| IV-19 | 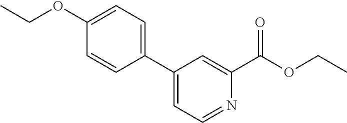 [SMILES: CCOC(=O)c1cc(-c2ccc(OCC)cc2)ccn1] |
| IV-20 | 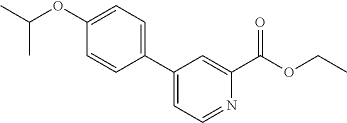 [SMILES: CCOC(=O)c1cc(-c2ccc(OC(C)C)cc2)ccn1] |
| IV-21 | 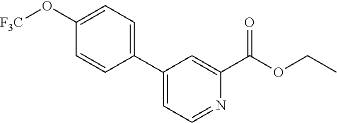 [SMILES: CCOC(=O)c1cc(-c2ccc(OC(F)(F)F)cc2)ccn1] |
| IV-22 | 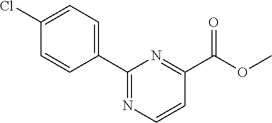 [SMILES: COC(=O)c1ccnc(-c2ccc(Cl)cc2)n1] |
| IV-23 | 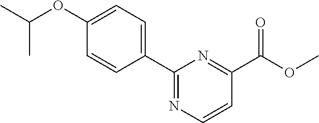 [SMILES: COC(=O)c1ccnc(-c2ccc(OC(C)C)cc2)n1] |
| IV-24 | 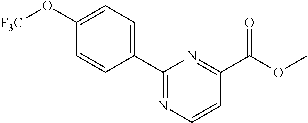 [SMILES: COC(=O)c1ccnc(-c2ccc(OC(F)(F)F)cc2)n1] |
| IV-25 | 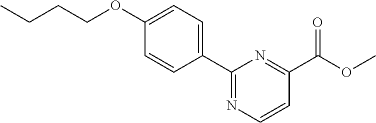 [SMILES: CCCCOc1ccc(-c2nccc(C(=O)OC)n2)cc1] |
| IV-26 | 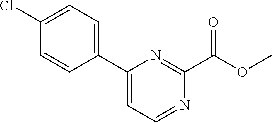 [SMILES: COC(=O)c1nccc(-c2ccc(Cl)cc2)n1] |
| IV-27 | 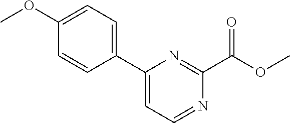 |
| IV-28 | 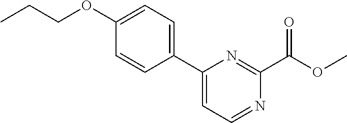 [SMILES: CCCOc1ccc(-c2ccnc(C(=O)OC)n2)cc1] |
| IV-29 | 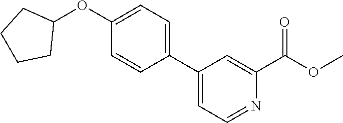 [SMILES: COC(=O)c1cc(-c2ccc(OC3CCCC3)cc2)ccn1] |
| IV-30 | 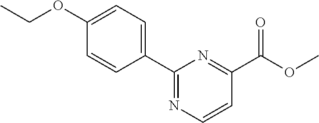 [SMILES: CCOc1ccc(-c2nccc(C(=O)OC)n2)cc1] |
| IV-31 | 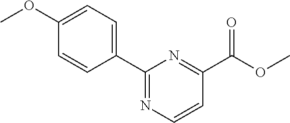 |
| IV-32 | 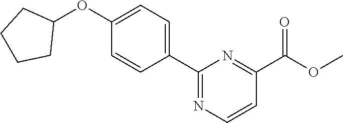 [SMILES: COC(=O)c1ccnc(-c2ccc(OC3CCCC3)cc2)n1] |
| IV-33 | 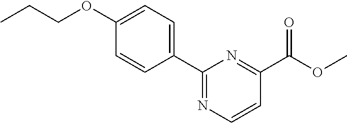 [SMILES: CCCOc1ccc(-c2nccc(C(=O)OC)n2)cc1] |
| IV-52 | 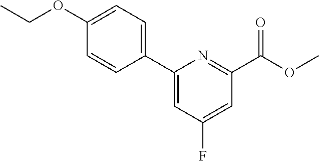 [SMILES: CCOc1ccc(-c2cc(F)cc(C(=O)OC)n2)cc1] |
| IV-53 | 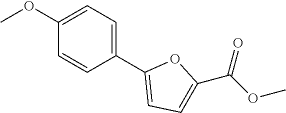 |
| IV-54 | 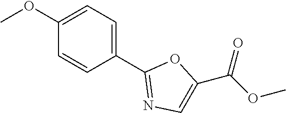 |
| IV-55 | 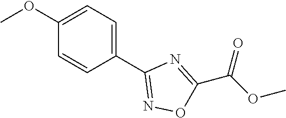 |
| IV-56 | 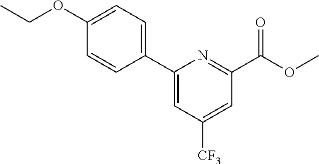 [SMILES: CCOc1ccc(-c2cc(C(F)(F)F)cc(C(=O)OC)n2)cc1] |
| IV-57 | 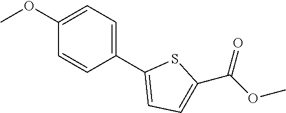 |
| IV-58 | 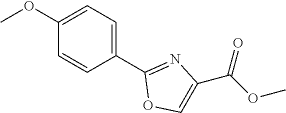 |
| IV-59 | 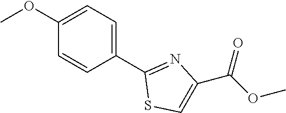 |

### Method IV-B:

### Intermediates IV-34-IV-43

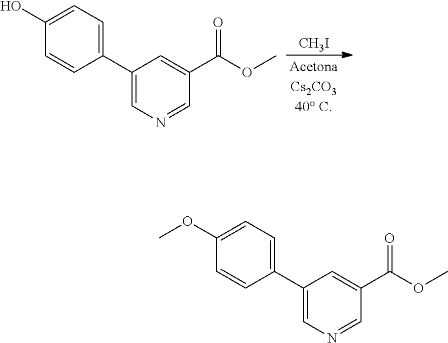

Into a 25-mL flask, 1.3 mmol of methyl 5-(4-hydroxyphenyl) nicotinate, 3 equivalents of cesium carbonate, and 9 mL of acetone were added. Subsequently, 2 equivalents of methyl iodide in 1 mL of acetone were added. The reaction mixture was maintained under stirring at 40° C. After total consumption of the starting reagent (monitored by CCF), the reaction mixture was diluted with H2O (25 mL) and extracted with AcOEt (3×25 mL). The organic phases were pooled, dried over anhydrous magnesium sulfate, filtered and concentrated. Purification was performed by column chromatography (n-Hexane/AcOEt: 90/10→70/30) affording the intermediate of interest (Table 2).

TABLE 2
Intermediates obtained, from the corresponding reagents, following
Method IV-B.

| Inter- | |
|---|---|
| mediate | Structure |
| IV-34 | 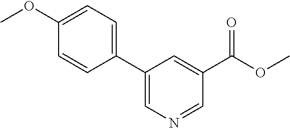 |
| IV-35 | 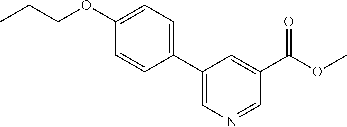 [SMILES: CCCOc1ccc(-c2cncc(C(=O)OC)c2)cc1] |
| IV-36 | 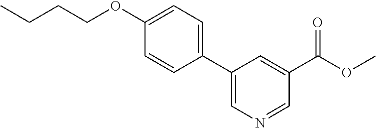 [SMILES: CCCCOc1ccc(-c2cncc(C(=O)OC)c2)cc1] |
| IV-37 | 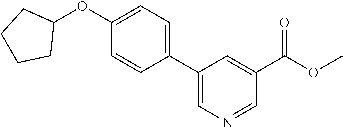 [SMILES: COC(=O)c1cncc(-c2ccc(OC3CCCC3)cc2)c1] |
| IV-38 | 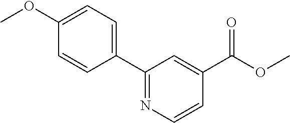 |
| IV-39 | 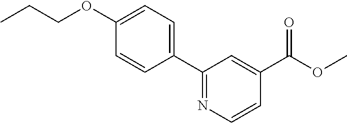 [SMILES: CCCOc1ccc(-c2cc(C(=O)OC)ccn2)cc1] |
| IV-40 | 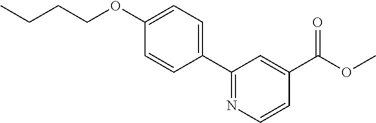 [SMILES: CCCCOc1ccc(-c2cc(C(=O)OC)ccn2)cc1] |
| IV-41 | 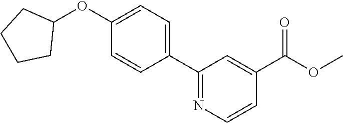 [SMILES: COC(=O)c1ccnc(-c2ccc(OC3CCCC3)cc2)c1] |
| IV-42 | 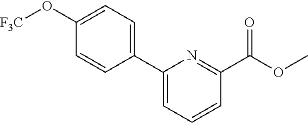 [SMILES: COC(=O)c1cccc(-c2ccc(OC(F)(F)F)cc2)n1] |
| IV-43 | 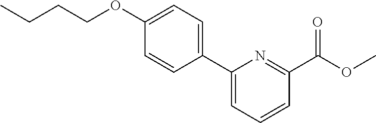 [SMILES: CCCCOc1ccc(-c2cccc(C(=O)OC)n2)cc1] |

### Method IV-C:

For the synthesis of intermediates IV-44 to IV-51 (Table 4), the synthesis of precursors IV-44i-IV-51i is required, which is obtained through two steps (IV-C-i and IV-C-ii), as described below.

### Step IV-C-i

### Precursors IV-44i-IV-51i

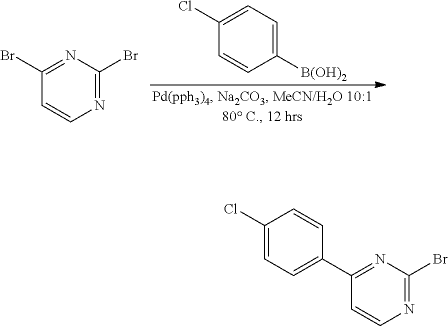 [SMILES: Brc1ccnc(Br)n1 ; OB(O)c1ccc(Cl)cc1 ; Clc1ccc(-c2ccnc(Br)n2)cc1]

Into a round bottom flask were added 12.6 mmol of 2,4-dibromopyrimidine, 12.8 mmol of 2,4-dibromopyrimidine, 1.26 mmol of Pd(dppf)Cl2CH2Cl2, 25.2 mmol of Na2CO3 20 mL of MeCN and 2 mL of H2O. Subsequently, the reaction mixture was allowed to stir at 80° C. After total consumption of the starting reagent (monitored by CCF), the reaction mixture was diluted with distilled water (30 mL) and extracted with AcOEt (3×10 mL). The organic phases were pooled, dried over anhydrous magnesium sulfate, filtered and concentrated to give the product, which was purified by column chromatography (n-hexane/AcOEt: 3/1→0/1) (Table 3).

For compound IV-47i, the solvent was replaced with a 10:1 dioxane/H2Omixture, using as catalyst Pd(dppf)Cl2CH2Cl2.

For compounds IV-48i to IV-51i, the starting reagent used was 2,4-dichloropyrimidine and the solvent a 10:1 dioxane/H2O mixture. Additionally, as catalyst was used Pd(PPh3) 4 and as base K2CO3.

TABLE 3
Precursors obtained, from the corresponding reagents, following step
IV-C-i of Method IV-C.

| Precursor | Structure |
|---|---|
| IV-44i | 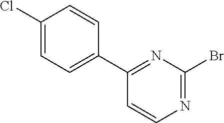 [SMILES: Clc1ccc(-c2ccnc(Br)n2)cc1] |
| IV-45i | 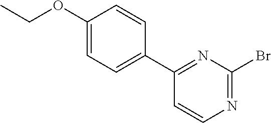 [SMILES: CCOc1ccc(-c2ccnc(Br)n2)cc1] |
| IV-46i | 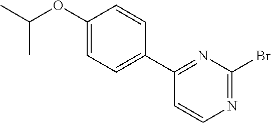 [SMILES: CC(C)Oc1ccc(-c2ccnc(Br)n2)cc1] |
| IV-47i | 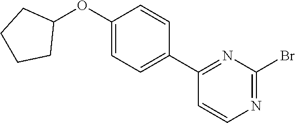 [SMILES: Brc1nccc(-c2ccc(OC3CCCC3)cc2)n1] |
| IV-48i | 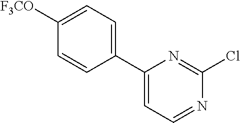 [SMILES: FC(F)(F)Oc1ccc(-c2ccnc(Cl)n2)cc1] |
| IV-49i | 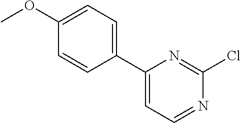 |
| IV-50i | 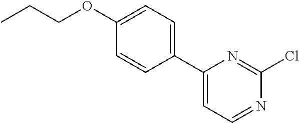 [SMILES: CCCOc1ccc(-c2ccnc(Cl)n2)cc1] |
| IV-51i | 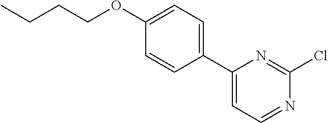 [SMILES: CCCCOc1ccc(-c2ccnc(Cl)n2)cc1] |

### Step IV-C-ii

### Intermediates IV-44-IV-51

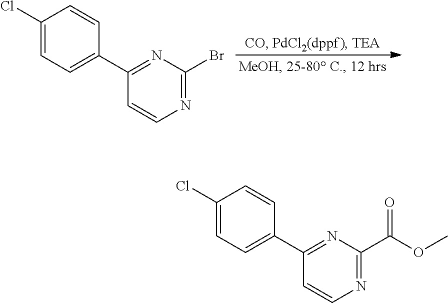

In a round bottom flask containing 7.4 mmol of 2-bromo-4-(4-chlorophenyl)pyrimidine in 5 mL of MeOH, 14.8 mmol of tea and 371 μmol ofPdCl2 (dppf) were added. Subsequently, the reaction mixture degassed, purged with CO and maintained, under CO atmosphere (50 Psi), stirring at 80° C. for 12 hours. After total consumption of the starting reagent (monitored by CCF), the reaction mixture concentrated to obtain the product, which was purified by column chromatography (n-hexane/AcOEt: 3/1→0/1) (Table 4).

TABLE 4
Precursors obtained, from the corresponding reagents, following step
IV-C-i of Method IV-C.

| Inter- | |
|---|---|
| mediate | Structure |
| IV-44 | 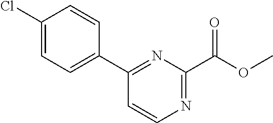 [SMILES: COC(=O)c1nccc(-c2ccc(Cl)cc2)n1] |
| IV-45 | 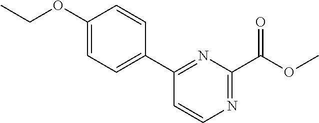 [SMILES: CCOc1ccc(-c2ccnc(C(=O)OC)n2)cc1] |
| IV-46 | 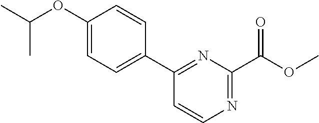 [SMILES: COC(=O)c1nccc(-c2ccc(OC(C)C)cc2)n1] |
| IV-47 | 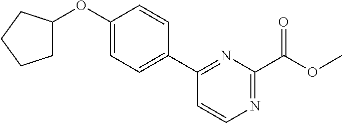 [SMILES: COC(=O)c1nccc(-c2ccc(OC3CCCC3)cc2)n1] |
| IV-48 | 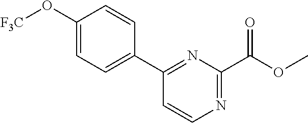 [SMILES: COC(=O)c1nccc(-c2ccc(OC(F)(F)F)cc2)n1] |
| IV-49 | 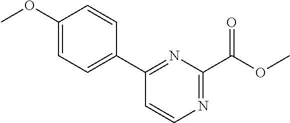 |
| IV-50 | 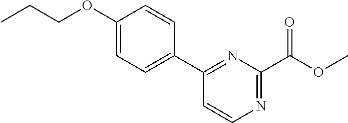 [SMILES: CCCOc1ccc(-c2ccnc(C(=O)OC)n2)cc1] |
| IV-51 | 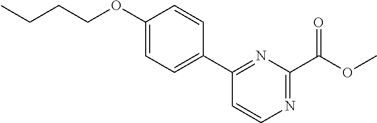 [SMILES: CCCCOc1ccc(-c2ccnc(C(=O)OC)n2)cc1] |

### Obtaining Compounds of Formula IV:

### Intermediates III-1-III-59

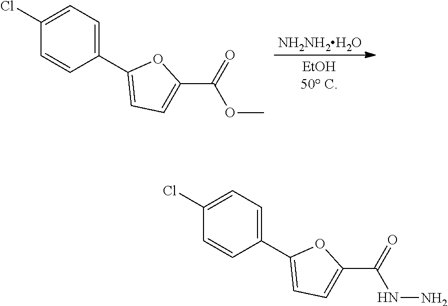

In a flask containing 8.8 mmol of methyl 5-(4-chlorophenyl) furan-2-carboxylate (intermediate IV-1), dissolved in 15 mL of ethanol, 3 equivalents of hydrazine hydroxide were added. The reaction medium was then subjected to heating to 50° C. When total consumption of starting ester by CCF was detected, the solvent was concentrated to half volume under reduced pressure, and 25 mL of cold distilled water was added, leading to precipitation of a solid which was filtered and washed with 25 mL of cold distilled water and 15 mL of n-hexane, providing the hydrazide of interest (Table 5).

TABLE 5
Intermediates obtained, from the corresponding reagents, following the
method described in obtaining the compounds of Formula III.

| Inter- | |
|---|---|
| me- | |
| diate | Structure |
| III-1 | 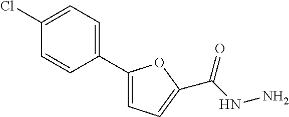 [SMILES: NNC(=O)c1ccc(-c2ccc(Cl)cc2)o1] |
| III-2 | 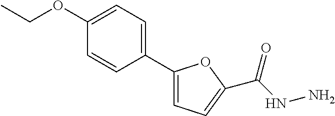 [SMILES: CCOc1ccc(-c2ccc(C(=O)NN)o2)cc1] |
| III-3 | 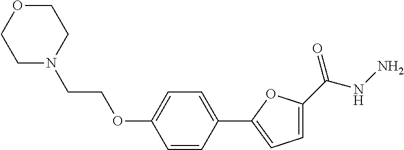 [SMILES: NNC(=O)c1ccc(-c2ccc(OCCN3CCOCC3)cc2)o1] |
| III-4 | 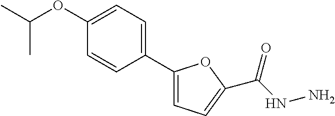 [SMILES: CC(C)Oc1ccc(-c2ccc(C(=O)NN)o2)cc1] |
| III-5 | 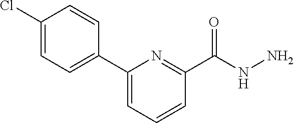 [SMILES: NNC(=O)c1cccc(-c2ccc(Cl)cc2)n1] |
| III-6 | 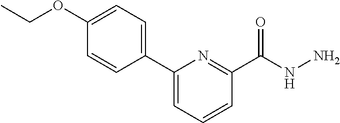 [SMILES: CCOc1ccc(-c2cccc(C(=O)NN)n2)cc1] |
| III-7 | 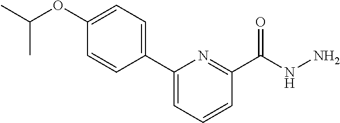 [SMILES: CC(C)Oc1ccc(-c2cccc(C(=O)NN)n2)cc1] |
| III-8 | 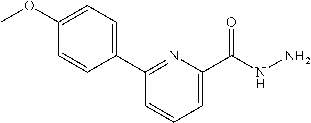 [SMILES: COc1ccc(-c2cccc(C(=O)NN)n2)cc1] |
| III-9 | 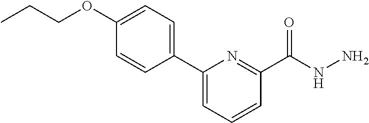 [SMILES: CCCOc1ccc(-c2cccc(C(=O)NN)n2)cc1] |
| III-10 | 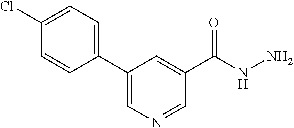 [SMILES: NNC(=O)c1cncc(-c2ccc(Cl)cc2)c1] |
| III-11 | 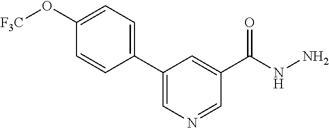 [SMILES: NNC(=O)c1cncc(-c2ccc(OC(F)(F)F)cc2)c1] |
| III-12 | 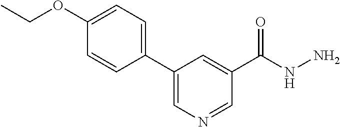 [SMILES: CCOc1ccc(-c2cncc(C(=O)NN)c2)cc1] |
| III-13 | 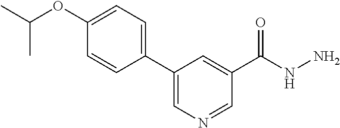 [SMILES: CC(C)Oc1ccc(-c2cncc(C(=O)NN)c2)cc1] |
| III-14 | 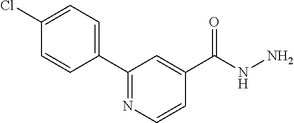 [SMILES: NNC(=O)c1ccnc(-c2ccc(Cl)cc2)c1] |
| III-15 | 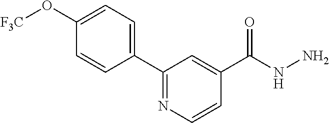 [SMILES: NNC(=O)c1ccnc(-c2ccc(OC(F)(F)F)cc2)c1] |
| III-16 | 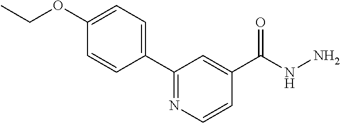 [SMILES: CCOc1ccc(-c2cc(C(=O)NN)ccn2)cc1] |
| III-17 | 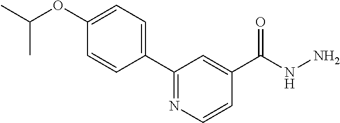 [SMILES: CC(C)Oc1ccc(-c2cc(C(=O)NN)ccn2)cc1] |
| III-18 | 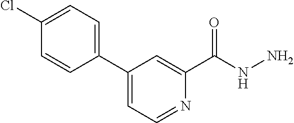 [SMILES: NNC(=O)c1cc(-c2ccc(Cl)cc2)ccn1] |
| III-19 | 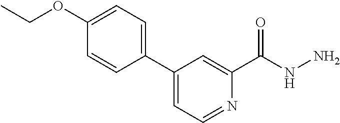 [SMILES: CCOc1ccc(-c2ccnc(C(=O)NN)c2)cc1] |
| III-20 | 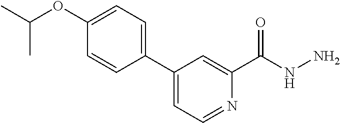 [SMILES: CC(C)Oc1ccc(-c2ccnc(C(=O)NN)c2)cc1] |
| III-21 | 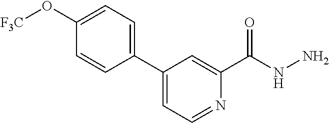 [SMILES: NNC(=O)c1cc(-c2ccc(OC(F)(F)F)cc2)ccn1] |
| III-22 | 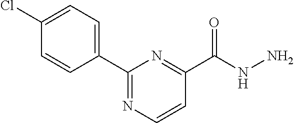 [SMILES: NNC(=O)c1ccnc(-c2ccc(Cl)cc2)n1] |
| III-23 | 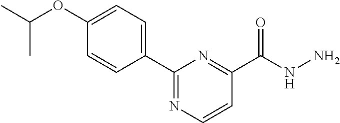 [SMILES: CC(C)Oc1ccc(-c2nccc(C(=O)NN)n2)cc1] |
| III-24 | 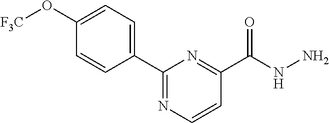 [SMILES: NNC(=O)c1ccnc(-c2ccc(OC(F)(F)F)cc2)n1] |
| III-25 | 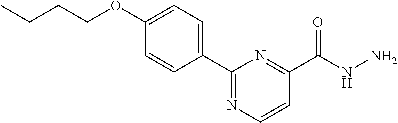 [SMILES: CCCCOc1ccc(-c2nccc(C(=O)NN)n2)cc1] |
| III-26 | 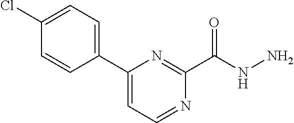 [SMILES: NNC(=O)c1nccc(-c2ccc(Cl)cc2)n1] |
| III-27 | 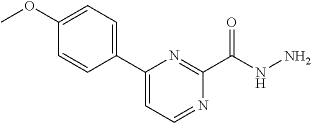 [SMILES: COc1ccc(-c2ccnc(C(=O)NN)n2)cc1] |
| III-28 | 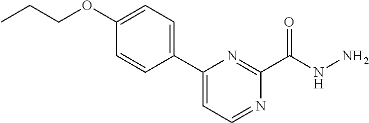 [SMILES: CCCOc1ccc(-c2ccnc(C(=O)NN)n2)cc1] |
| III-29 | 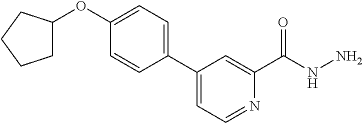 [SMILES: NNC(=O)c1cc(-c2ccc(OC3CCCC3)cc2)ccn1] |
| III-30 | 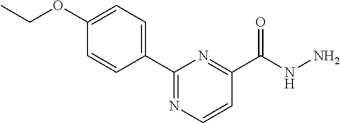 [SMILES: CCOc1ccc(-c2nccc(C(=O)NN)n2)cc1] |
| III-31 | 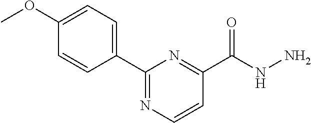 [SMILES: COc1ccc(-c2nccc(C(=O)NN)n2)cc1] |
| III-32 | 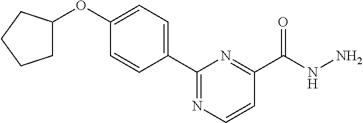 [SMILES: NNC(=O)c1ccnc(-c2ccc(OC3CCCC3)cc2)n1] |
| III-33 | 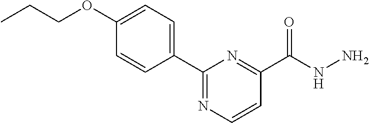 [SMILES: CCCOc1ccc(-c2nccc(C(=O)NN)n2)cc1] |
| III-34 | 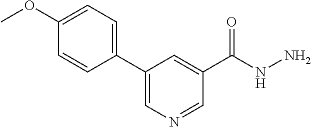 [SMILES: COc1ccc(-c2cncc(C(=O)NN)c2)cc1] |
| III-35 | 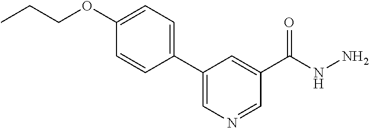 [SMILES: CCCOc1ccc(-c2cncc(C(=O)NN)c2)cc1] |
| III-36 | 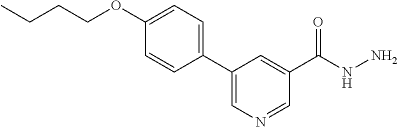 [SMILES: CCCCOc1ccc(-c2cncc(C(=O)NN)c2)cc1] |
| III-37 | 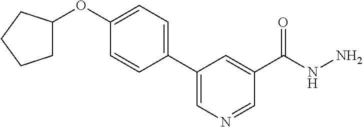 [SMILES: NNC(=O)c1cncc(-c2ccc(OC3CCCC3)cc2)c1] |
| III-38 | 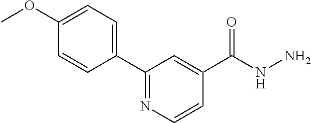 [SMILES: COc1ccc(-c2cc(C(=O)NN)ccn2)cc1] |
| III-39 | 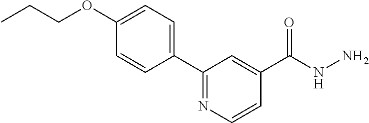 [SMILES: CCCOc1ccc(-c2cc(C(=O)NN)ccn2)cc1] |
| III-40 | 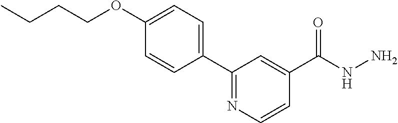 [SMILES: CCCCOc1ccc(-c2cc(C(=O)NN)ccn2)cc1] |
| III-41 | 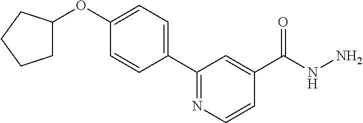 [SMILES: NNC(=O)c1ccnc(-c2ccc(OC3CCCC3)cc2)c1] |
| III-42 | 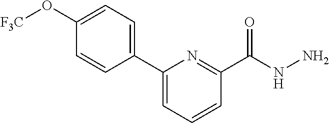 [SMILES: NNC(=O)c1cccc(-c2ccc(OC(F)(F)F)cc2)n1] |
| III-43 | 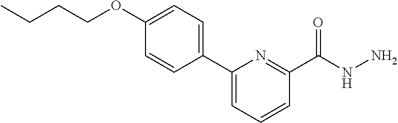 [SMILES: CCCCOc1ccc(-c2cccc(C(=O)NN)n2)cc1] |
| III-44 | 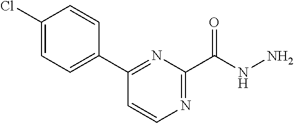 [SMILES: NNC(=O)c1nccc(-c2ccc(Cl)cc2)n1] |
| III-45 | 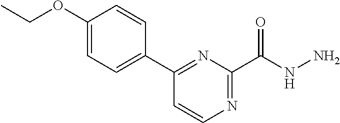 [SMILES: CCOc1ccc(-c2ccnc(C(=O)NN)n2)cc1] |
| III-46 | 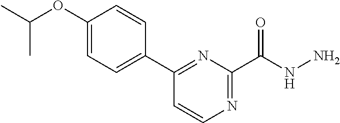 [SMILES: CC(C)Oc1ccc(-c2ccnc(C(=O)NN)n2)cc1] |
| III-47 | 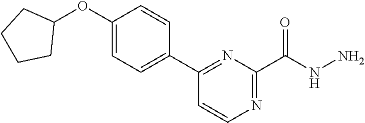 [SMILES: NNC(=O)c1nccc(-c2ccc(OC3CCCC3)cc2)n1] |
| III-48 | 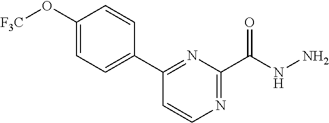 [SMILES: NNC(=O)c1nccc(-c2ccc(OC(F)(F)F)cc2)n1] |
| III-49 | 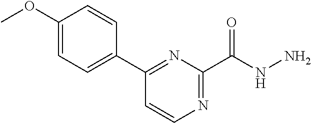 [SMILES: COc1ccc(-c2ccnc(C(=O)NN)n2)cc1] |
| III-50 | 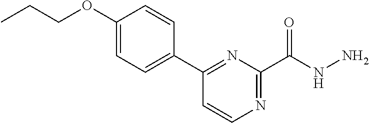 [SMILES: CCCOc1ccc(-c2ccnc(C(=O)NN)n2)cc1] |
| III-51 | 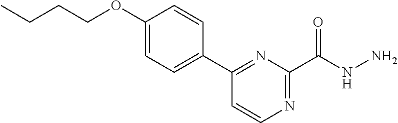 [SMILES: CCCCOc1ccc(-c2ccnc(C(=O)NN)n2)cc1] |
| III-52 | 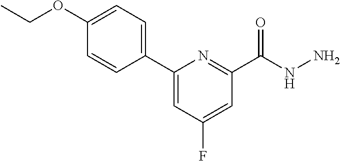 [SMILES: CCOc1ccc(-c2cc(F)cc(C(=O)NN)n2)cc1] |
| III-53 | 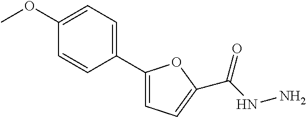 [SMILES: COc1ccc(-c2ccc(C(=O)NN)o2)cc1] |
| III-54 | 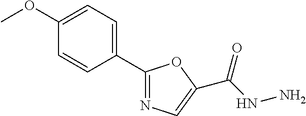 [SMILES: COc1ccc(-c2ncc(C(=O)NN)o2)cc1] |
| III-55 | 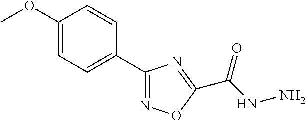 [SMILES: COc1ccc(-c2noc(C(=O)NN)n2)cc1] |
| III-56 | 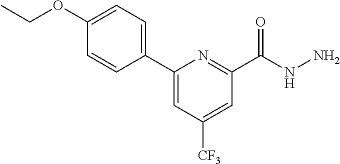 [SMILES: CCOc1ccc(-c2cc(C(F)(F)F)cc(C(=O)NN)n2)cc1] |
| III-57 | 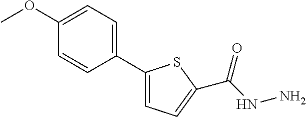 [SMILES: COc1ccc(-c2ccc(C(=O)NN)s2)cc1] |
| III-58 | 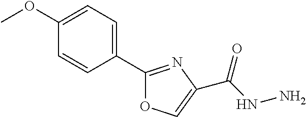 [SMILES: COc1ccc(-c2nc(C(=O)NN)co2)cc1] |
| III-59 | 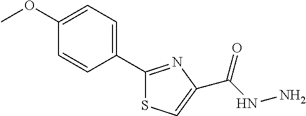 [SMILES: COc1ccc(-c2nc(C(=O)NN)cs2)cc1] |

### EXAMPLES

The following examples, described in detail, serve to illustrate embodiments of the present invention without, however, having any limiting character to the scope of protection thereof.

The compound(s) of Formula (I) of the present invention were obtained from the condensation of the intermediates of Formula III, obtained according to previously described methodologies without being limited thereto, with commercial aldehydes of Formula II, according to General Scheme 1.

### Example 1. Obtaining Compounds of Formula (I)

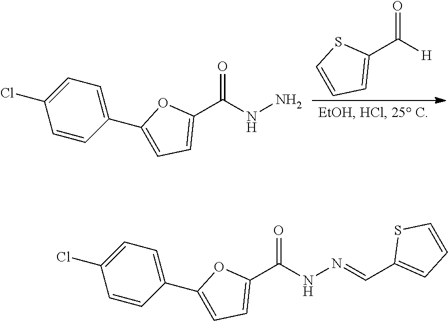 [SMILES: NNC(=O)c1ccc(-c2ccc(Cl)cc2)o1 ; O=Cc1cccs1 ; O=C(N/N=C/c1cccs1)c1ccc(-c2ccc(Cl)cc2)o1]

In a flask containing 1 mmol of 5-(4-chlorophenyl) furan-2-carbohydrazide (intermediate III-1), dissolved in 10 mL of ethanol, catalytic amounts of concentrated HCl were added. Subsequently, 1.1 mmol of thiophene-2-carbaldehyde dissolved in 2 mL of ethanol was added. The reaction medium remained under agitation until total consumption of the starting material (detected by CCF). After concentration to half the volume of the reaction medium under reduced pressure, 10 mL of ice-cold distilled water was added while keeping the mixture in an ice bath. After filtration and washing with 15 mL of ice water and 15 mL of hexane, compound 1 was obtained (Table 6).

TABLE 6
Compounds obtained, from the corresponding reagents, following the
method described for formation of Compound 1.

| Compound | | 1H-NMR/ |
|---|---|---|
| No. | Structure | MS m/z [M + H]+ |
| 1 | 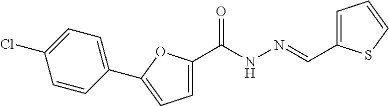 [SMILES: O=C(N/N=C/c1cccs1)c1ccc(-c2ccc(Cl)cc2)o1] | 400 MHz, DMSO-d6 δ 11.79 (s, 1H), 8.74 (s, 1H), 7.96 (d, 2H), 7.69 (d, 1H), 7.56 (d, 2H), 7.49 (d, 1H), 7.37 (d, 1H), 7.22 (d, 1H), 7.16 (t, 1H). [M + H]+: 331.7. |
| | (E)-5-(4-chlorophenyl)-N′-(thiophen-2- | |
| | ylmethylene)furan-2-carbohydrazide | |
| | (Compound 1) | |
| 2 | 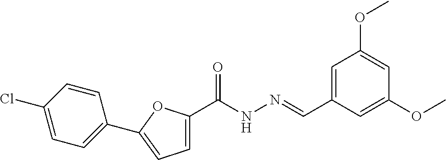 [SMILES: COc1cc(/C=N/NC(=O)c2ccc(-c3ccc(Cl)cc3)o2)cc(OC)c1] | 400 MHz, DMSO-d6 δ 11.83 (s, 1H), 8.44 (s, 1H), 7.98 (d, J = 7.1 Hz, 2H), 7.58 (d, J = 8.3 Hz, 2H), 7.40 (s, 1H), 7.24 (d, J = 2.7 Hz, 1H), 6.89 (s, 2H), 6.59 (s, 1H), 3.80 (s, 6H) |
| | (E)-5-(4-chlorophenyl)-N′-(3,5- | |
| | dimethoxybenzylidene)furan-2- | |
| | carbohydrazide | |
| 3 | 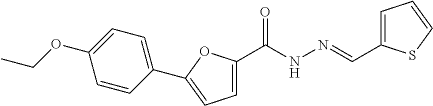 [SMILES: CCOc1ccc(-c2ccc(C(=O)N/N=C/c3cccs3)o2)cc1] | 400 MHz, DMSO-d6 δ 11.70 (s, 1H), 8.73 (s, 1H), 7.85 (d, J = 6.6 Hz, 2H), 7.68 (d, J = 5.0 Hz, 1H), 7.48 (d, J = 3.3 Hz, 1H), 7.33 (s, 1H), 7.16 (dd, J = 5.0, 3.6 Hz, 1H), 7.06-7.01 (m, 3H), 4.11-4.07 (m, 1H), 1.35 (t, J = 5.0 Hz, 6H). [M + H]+: 341.5. |
| | (E)-5-(4-ethoxyphenyl)-N′-(thiophen-2- | |
| | ylmethylene)furan-2-carbohydrazide | |
| 4 | 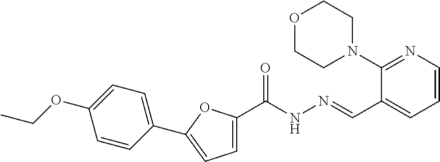 [SMILES: CCOc1ccc(-c2ccc(C(=O)N/N=C/c3cccnc3N3CCOCC3)o2)cc1] | 400 MHz, DMSO-d6 δ 11.84 (s, 1H), 8.57 (s, 1H), 8.29 (dd, J = 4.9, 1.9 Hz, 1H), 8.10 (dd, J = 7.6, 1.9 Hz, 1H), 7.82 (d, J = 8.2 Hz, 2H), 7.34 (s, 1H), 7.09 (dd, J = 7.7, 4.7 Hz, 1H), 7.04-6.97 (m, 3H), 4.05 (q, J = 6.9 Hz, 2H), 3.77 (t, J = 4.5 Hz, 4H), 3.11 (t, J = 4.6 Hz, 4H), 1.31 (t, J = 6.9 Hz, 3H). |
| | (E)-5-(4-ethoxyphenyl)-N′-((2- | |
| | morpholinopyridin-3-yl)methylene)furan- | |
| | 2-carbohydrazide | |
| 5 | 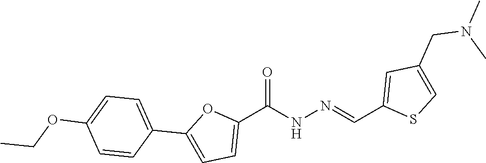 [SMILES: CCOc1ccc(-c2ccc(C(=O)N/N=C/c3cc(CN(C)C)cs3)o2)cc1] | 400 MHz, DMSO-d6 δ 8.78 (s, 1H), 7.96 (d, J = 8.2 Hz, 2H), 7.57-7.37 (m, 3H), 7.12 (dd, J = 15.6, 5.9 Hz, 3H), 4.19 (q, J = 6.9 Hz, 2H), 3.47 (m, 2H), 2.25 (s, 6H), 1.45 (t, J = 6.9 Hz, 3H). [M + H]+: 398.4. |
| | (E)-N′-((4- | |
| | ((dimethylamino)methyl)thiophen-2- | |
| | yl)methylene)-5-(4-ethoxyphenyl)furan-2- | |
| | carbohydrazide | |
| 6 | 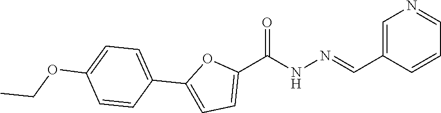 [SMILES: CCOc1ccc(-c2ccc(C(=O)N/N=C/c3cccnc3)o2)cc1] | 400 MHz, DMSO-d6 δ 11.89 (s, 1H), 8.83 (s, 1H), 8.59 (d, J = 4.8 Hz, 1H), 8.52 (s, 1H), 8.12 (d, J = 8.0 Hz, 1H), 7.83 (s, 2H), 7.47 (dd, J = 7.9, 4.7 Hz, 1H), 7.35 (s, 1H), 7.01 (d, J = 8.7 Hz, 3H), 4.06 (q, J = 7.0 Hz, 2H), 1.31 (t, J = 7.0 Hz, 3H). |
| | (E)-5-(4-ethoxyphenyl)-N′-(pyridin-3- | |
| | ylmethylene)furan-2-carbohydrazide | |
| 7 | 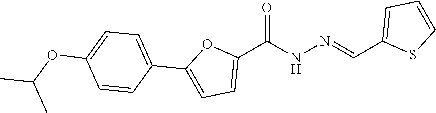 [SMILES: CC(C)Oc1ccc(-c2ccc(C(=O)N/N=C/c3cccs3)o2)cc1] | 500 MHz, DMSO-d6 δ 11.70 (s, 1H), 8.73 (s, 1H), 7.85 (d, J = 6.6 Hz, 2H), 7.68 (d, J = 5.0 Hz, 1H), 7.48 (d, J = 3.3 Hz, 1H), 7.33 (s, 1H), 7.16 (dd, J = 5.0, 3.6 Hz, 1H), 7.04-6.99 (m, 3H), 4.72-4.68 (m, 1H), 1.29 (d, J = 6.0 Hz, 6H). [M + H]+: 355.5. |
| | (E)-5-(4-isopropoxyphenyl)-N′- | |
| | (thiophen-2-ylmethylene)furan-2- | |
| | carbohydrazide | |
| 8 | 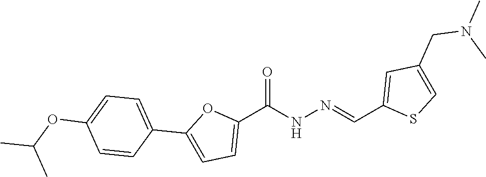 [SMILES: CC(C)Oc1ccc(-c2ccc(C(=O)N/N=C/c3cc(CN(C)C)cs3)o2)cc1] | 400 MHz, DMSO-d6 δ 11.71 (s, 1H), 8.67 (s, 1H), 7.85 (d, J = 7.8 Hz, 2H), 7.42 (s, 1H), 7.37 (s, 1H), 7.33 (s, 1H), 7.04-6.98 (m, 3H), 4.70 (sep, J = 6.1 Hz, 1H), 3.38 (s, 2H), 2.15 (s, 6H), 1.29 (d, J = 6.0 Hz, 6H). [M + H]+: 412.6. |
| | (E)-N′-((4- | |
| | ((dimethylamino)methyl)thiophen-2- | |
| | yl)methylene)-5-(4- | |
| | isopropoxyphenyl)furan-2- | |
| | carbohydrazide | |
| 9 | 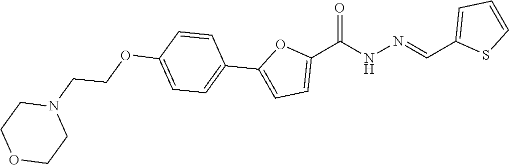 [SMILES: O=C(N/N=C/c1cccs1)c1ccc(-c2ccc(OCCN3CCOCC3)cc2)o1] | 400 MHz, DMSO-d6 δ 11.79 (s, 1H), 8.81 (s, 1H), 8.01-7.84 (m, 2H), 7.77 (d, J = 5.0 Hz, 1H), 7.56 (d, J = 3.6 Hz, 1H), 7.41 (s, 1H), 7.24 (t, J = 4.4 Hz, 1H), 7.15 (d, J = 8.4 Hz, 2H), 7.10 (d, J = 3.5 Hz, 1H), 4.23 (t, J = 5.7 Hz, 2H), 3.67 (t, J = 4.7 Hz, 4H), 2.79 (t, J = 5.6 Hz, 2H). |
| | (E)-5-(4-(2-morpholinoethoxy)phenyl)-N′- | |
| | (thiophen-2-ylmethylene)furan-2- | |
| | carbohydrazide | |
| 10 | 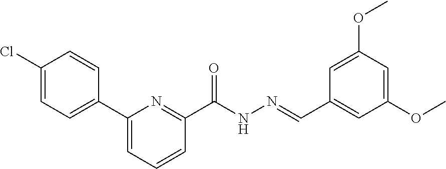 [SMILES: COc1cc(/C=N/NC(=O)c2cccc(-c3ccc(Cl)cc3)n2)cc(OC)c1] | 400 MHz, DMSO-d6 δ 11.95 (s, 1H), 8.66 (s, 1H), 8.39 (d, 2H), 8.24 (d, 1H), 8.13 (t, 1H), 8.07 (d, 1H), 7.59 (d, 2H), 6.92 (s, 2H), 6.60 (s, 1H), 3.81 (s, 6H). [M + H]+: 396.7. |
| | (E)-6-(4-chlorophenyl)-N′-(3,5- | |
| | dimethoxybenzylidene)picolinohydrazide | |
| 11 | 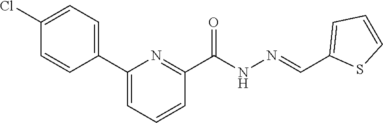 [SMILES: O=C(N/N=C/c1cccs1)c1cccc(-c2ccc(Cl)cc2)n1] | 400 MHz, DMSO-d6 δ 11.92 (s, 1H), 8.96 (s, 1H), 8.40 (d, 2H), 8.24 (d, 1H), 8.13 (t, 1H), 8.08 (d, 1H), 7.71 (d, 1H), 7.59 (d, 2H), 7.50 (d, 1H), 7.17 (t, 1H). [M + H]+: 342.0. |
| | (E)-6-(4-chlorophenyl)-N′-(thiophen-2- | |
| | ylmethylene)picolinohydrazide | |
| 12 | 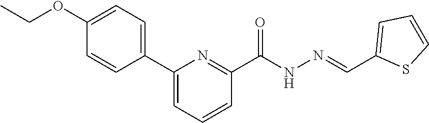 [SMILES: CCOc1ccc(-c2cccc(C(=O)N/N=C/c3cccs3)n2)cc1] | 400 MHz, DMSO-d6 δ 11.88 (s, 1H), 8.95 (s, 1H), 8.30 (d, 2H), 8.13 (d, 1H), 8.05 (t, 1H), 7.97 (d, 1H), 7.70 (d, 1H), 7.49 (d, 1H), 7.17 (t, 1H), 7.05 (d, 2H), 4.10 (q, 2H), 1.36 (t, 3H). [M + H]+: 352.6. |
| | (E)-6-(4-ethoxyphenyl)-N′-(thiophen-2- | |
| | ylmethylene)picolinohydrazide | |
| 13 | 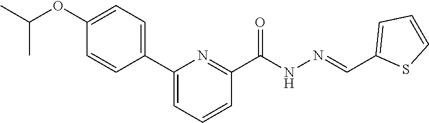 [SMILES: CC(C)Oc1ccc(-c2cccc(C(=O)N/N=C/c3cccs3)n2)cc1] | 400 MHz, DMSO-d6 δ 11.86 (s, 1H), 8.94 (s, 1H), 8.13 (d, 1H), 8.05 (t, 1H), 7.96 (d, 1H), 7.70 (d, 1H), 7.17 (t, 1H), 7.04 (d, 2H), 4.73 (m, 1H), 1.31 (d, 6H). |
| | (E)-6-(4-isopropoxyphenyl)-N′-(thiophen- | |
| | 2-ylmethylene)picolinohydrazide | |
| 14 | 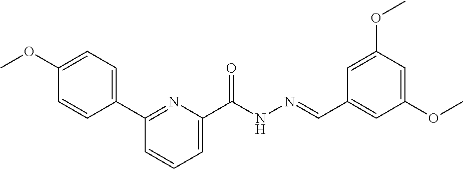 | 400 MHz, DMSO-d6 δ 11.90 (s, 1H), 8.66 (s, 1H), 8.32 (d, J = 8.9 Hz, 2H), 8.16 (d, J = 9.2 Hz, 1H), 8.06 (t, J = 7.8 Hz, 1H), 8.00 (d, J = 8.8 Hz, 1H), 7.08 (d, J = 8.9 Hz, 2H), 6.92 (d, J = 2.3 Hz, 2H), 6.60 (t, J = 2.3 Hz, 1H), 3.85 (s, 3H), 3.81 (s, 6H). [M + H]+: 392.5. |
| | (E)-N′-(3,5-dimethoxybenzylidene)-6-(4- | |
| | methoxyphenyl)picolinohydrazide | |
| 15 | 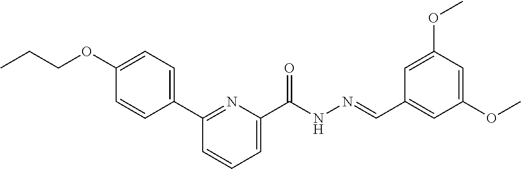 [SMILES: CCCOc1ccc(-c2cccc(C(=O)N/N=C/c3cc(OC)cc(OC)c3)n2)cc1] | 400 MHz, DMSO-d6 δ 11.90 (s, 1H), 8.66 (s, 1H), 8.31 (d, J = 8.9 Hz, 2H), 8.15 (dd, J = 7.9, 1.2 Hz, 1H), 8.06 (t, J = 7.8 Hz, 1H), 7.99 (t, J = 7.8 Hz, 1H), 7.07 (d, J = 8.9 Hz, 2H), 6.92 (d, J = 2.3 Hz, 2H), 6.60 (t, J = 2.3 Hz, 1H), 4.02 (t, J = 6.6 Hz, 2H), 3.81 (s, 6H), 1.77 (m, 2H), 1.01 (t, J = 7.4 Hz, 3H). [M + H]+: 420.1. |
| | (E)-N′-(3,5-dimethoxybenzylidene)-6-(4- | |
| | propoxyphenyl)picolinohydrazide | |
| 16 | 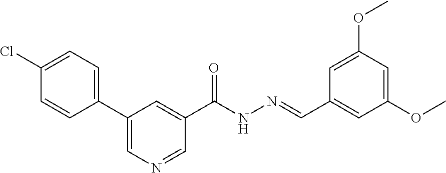 [SMILES: COc1cc(/C=N/NC(=O)c2cncc(-c3ccc(Cl)cc3)c2)cc(OC)c1] | 500 MHz, DMSO-d6 δ 12.09 (s, 1H), 9.10 (d, J = 1.9 Hz, 1H), 9.04 (d, J = 1.6 Hz, 1H), 8.51 (s, 1H), 8.38 (s, 1H), 7.88 (d, J = 8.5 Hz, 2H), 7.62 (d, J = 8.5 Hz, 2H), 6.91 (d, J = 2.0 Hz, 2H), 6.60 (s, 1H), 3.80 (s, 6H). [M + H]+: 396.0. |
| | (E)-5-(4-chlorophenyl)-N′-(3,5- | |
| | dimethoxybenzylidene)nicotinohydrazide | |
| | (Compound 16); | |
| 17 | 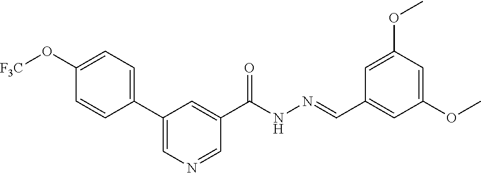 [SMILES: COc1cc(/C=N/NC(=O)c2cncc(-c3ccc(OC(F)(F)F)cc3)c2)cc(OC)c1] | 500 MHz, DMSO-d6 12.01 (s, 1H); 9.11 (d, J = 1.6, 1H); 9.06 (d, J = 1.6, 1H); 8.52 (bs, 1H); 8.38 (s, 1H); 7.97 (d, J = 8.4, 2H); 7.56 (d, J = 8.4, 2H); 6.91 (s, 2H); 6.60 (s, 1H); 3.80 (s, 6H). [M + H]+: 446.6. |
| | (E)-N′-(3,5-dimethoxybenzylidene)-5-(4- | |
| | (trifluoromethoxy)phenyl)nicotinohydrazide | |
| 18 | 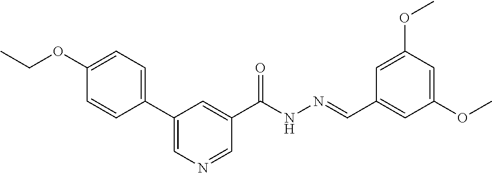 [SMILES: CCOc1ccc(-c2cncc(C(=O)N/N=C/c3cc(OC)cc(OC)c3)c2)cc1] | 500 MHz, DMSO-d6 δ 12.01 (s, 1H); 9.04 (d, J = 1.6, 1H); 8.97 (d, J = 1.6, 1H); 8.44 (sl, 1H); 8.38 (s, 1H); 7.77 (d, J = 8.4, 2H); 7.09 (d, J = 8.8, 2H); 6.90 (s, 2H); 6.60 (s, 1H); 4.10 (q, J = 6.8, 2H); 3.80 (s, 6H); 1.36 (t, J = 7.2, 3H). [M + H]+: 406.4. |
| | (E)-N′-(3,5-dimethoxybenzylidene)-5-(4- | |
| | ethoxyphenyl)nicotinohydrazide | |
| | (Compound 18); | |
| 19 | 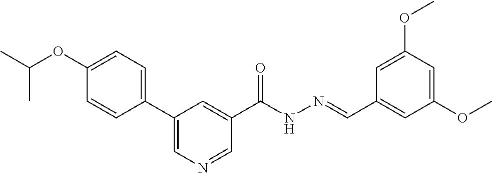 [SMILES: COc1cc(/C=N/NC(=O)c2cncc(-c3ccc(OC(C)C)cc3)c2)cc(OC)c1] | 500 MHz, DMSO-d6 δ 12.06 (1H, s); 9.04 (1H, d, J = 1.6); 8.97 (1H, d, J = 1.6); 8.44 (1H, sl); 8.38 (1H, s); 7.75 (2H, d, J = 8.8); 7.08 (2H, d, J = 8.8); 6.90 (2H, sl); 6.60 (1H, s); 4,71 (1H, sep, J = 6); 3.80 (6H, s); 1.29 (6H, d, J = 6). [M + H]+: 420.5. |
| | (E)-N′-(3,5-dimethoxybenzylidene)-5-(4- | |
| | isopropoxyphenyl)nicotinohydrazide | |
| 20 | 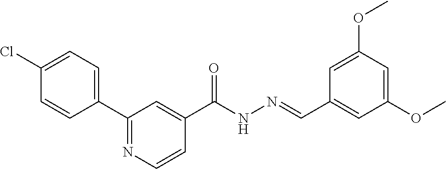 [SMILES: COc1cc(/C=N/NC(=O)c2ccnc(-c3ccc(Cl)cc3)c2)cc(OC)c1] | 400 MHz, DMSO-d6 δ 12.14 (s, 1H), 8.86 (d, J = 5.0 Hz, 1H), 8.41 (s, 1H), 8.37 (s, 1H), 8.20 (d, J = 8.6 Hz, 2H), 7.82-7.77 (m, 1H), 7,61 (d, J = 8.6 Hz, 2H), 6.91 (d, J = 2.1 Hz, 2H), 6.61 (t, J = 2.1 Hz, 1H), 3.80 (s, 6H). [M + H]+: 396.6. |
| | (E)-2-(4-chlorophenyl)-N′-(3,5- | |
| | dimethoxybenzylidene)isonicotinohydrazide | |
| 21 | 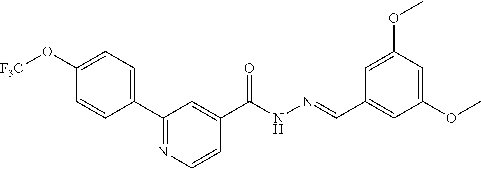 [SMILES: COc1cc(/C=N/NC(=O)c2ccnc(-c3ccc(OC(F)(F)F)cc3)c2)cc(OC)c1] | 400 MHz, DMSO-d6 δ 12.11 (s, 1H), 8.79 (d, J = 5.0 Hz, 1H), 8.41 (s, 1H), 8.26 (s, 1H), 8.10 (d, J = 8.8 Hz, 2H), 7.71-7.66 (m, 1H), 7.06 (d, J = 8.8 Hz, 2H), 6.91 (d, J = 2.2 Hz, 2H), 6.61 (t, J = 2.1 Hz, 1H), 4.71 (h, J = 6.0 Hz, 1H), 3.80 (s, 6H), 1.30 (d, J = 6.0 Hz, 6H). [M + H]+: 445.8. |
| | (E)-N′-(3,5-dimethoxybenzylidene)-2-(4- | |
| | (trifluoromethoxy)phenyl)isonicotinohydrazide | |
| 22 | 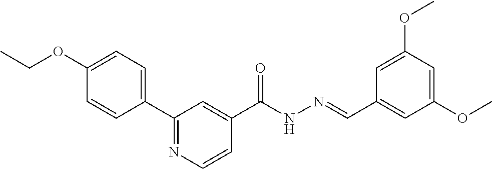 [SMILES: CCOc1ccc(-c2cc(C(=O)N/N=C/c3cc(OC)cc(OC)c3)ccn2)cc1] | 400 MHz, DMSO-d6 δ 12.11 (s, 1H), 8.80 (d, J = 4.9 Hz, 1H), 8.41 (s, 1H), 8.27 (s, 1H), 8.11 (d, J = 8.8 Hz, 2H), 7.71-7.66 (m, 1H), 7.07 (d, J = 8.8 Hz, 2H), 6.91 (d, J = 2.1 Hz, 2H), 6.61 (t, J = 2.1 Hz, 1H), 4,11 (q, J = 6.9 Hz, 2H), 3.80 (s, 6H), 1.36 (t, J = 7.0 Hz, 3H). [M + H]+: 406.3. |
| | (E)-N′-(3,5-dimethoxybenzylidene)-2-(4- | |
| | ethoxyphenyl)isonicotinohydrazide | |
| 23 | 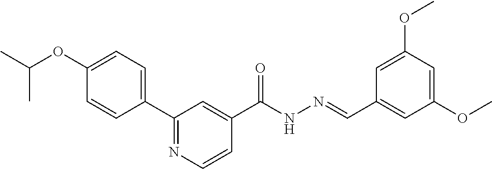 [SMILES: COc1cc(/C=N/NC(=O)c2ccnc(-c3ccc(OC(C)C)cc3)c2)cc(OC)c1] | 400 MHz, DMSO-d6 δ 12.11 (s, 1H), 8.79 (d, J = 5.0 Hz, 1H), 8.41 (s, 1H), 8.26 (s, 1H), 8.10 (d, J = 8.8 Hz, 2H), 7.71-7.66 (m, 1H), 7.06 (d, J = 8.8 Hz, 2H), 6.91 (d, J = 2.2 Hz, 2H), 6.61 (t, J = 2.1 Hz, 1H), 4.71 (h, J = 6.0 Hz, 1H), 3.80 (s, 6H), 1.30 (d, J = 6.0 Hz, 6H). [M + H]+: 420.0. |
| | (E)-N′-(3,5-dimethoxybenzylidene)-2-(4- | |
| | isopropoxyphenyl)isonicotinohydrazide | |
| 24 | 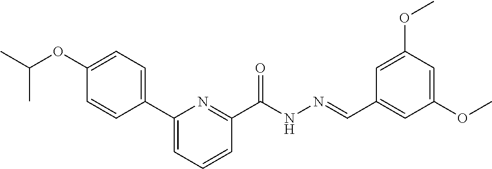 [SMILES: COc1cc(/C=N/NC(=O)c2cccc(-c3ccc(OC(C)C)cc3)n2)cc(OC)c1] | 400 MHz, DMSO-d6 δ 11.91 (s, 1H), 8.65 (s, 1H), 8.30 (d, J = 8.8 Hz, 2H), 8.16 (d, J = 8.0 Hz, 1H), 8.06 (t, J = 7.8 Hz, 1H), 7.99 (d, J = 7.4 Hz, 1H), 7.06 (d, J = 8.8 Hz, 2H), 6.92 (d, J = 2.1 Hz, 2H), 6.60 (s, 1H), 4.74 (m, 1H), 3.81 (s, 6H), 1.31 (d, J = 6.0 Hz, 6H). |
| | (E)-N′-(3,5-dimethoxybenzylidene)-6-(4- | |
| | isopropoxyphenyl)picolinohydrazide | |
| 25 | 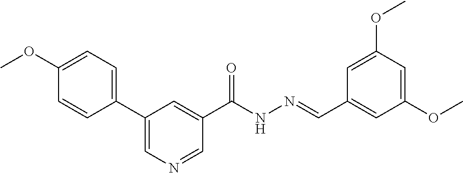 | 400 MHz, DMSO-d6 δ 12.18 (s, 1H), 9.08 (d, J = 2.0 Hz, 1H), 9.00 (d, J = 1.9 Hz, 1H), 8.53 (s, 1H), 8.41 (s, 1H), 7.81 (H-11, d, J = 8.7 Hz, 2H), 7.12 (H-12, d, J = 8.8 Hz, 2H), 6.91 (d, J = 2.2 Hz, 2H), 6.60 (t, J = 2.3 Hz, 1H), 3.83 (s, 3H), 3.80 (H-29, s, 6H). [M + H]+: 392.5. |
| | (E)-N′-(3,5-dimethoxybenzylidene)-5-(4- | |
| | methoxyphenyl)nicotinohydrazide | |
| 26 | 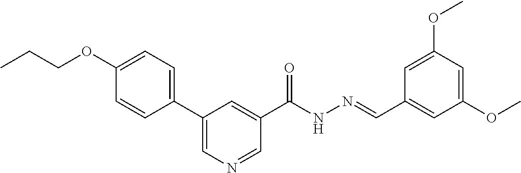 [SMILES: CCCOc1ccc(-c2cncc(C(=O)N/N=C/c3cc(OC)cc(OC)c3)c2)cc1] | 400 MHz, DMSO-d6 δ 12.10 (s, 1H), 9.05 (d, J = 2.2 Hz, 1H), 8.97 (d, J = 1.9 Hz, 1H), 8.46 (t, J = 2.1 Hz, 1H), 8.38 (s, 1H), 7.78 (d, J = 8.8 Hz, 2H), 7.10 (d, J = 8.8 Hz, 2H), 6.91 (d, J = 2.2 Hz, 2H), 6.60 (t, J = 2.3 Hz, 1H), 4.00 (t, J = 6.5 Hz, 2H), 3.80 (s, 6H), 1.76 (q, J = 6.9 Hz, 2H), 1.00 (t, J = 7.4 Hz, 3H). [M + H]+: 420.4. |
| | (E)-N′-(3,5-dimethoxybenzylidene)-5-(4- | |
| | propoxyphenyl)nicotinohydrazide | |
| 27 | 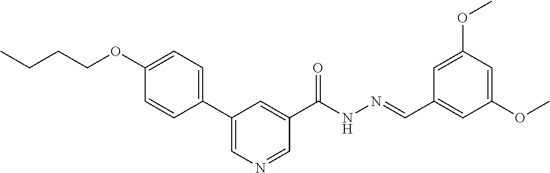 [SMILES: CCCCOc1ccc(-c2cncc(C(=O)N/N=C/c3cc(OC)cc(OC)c3)c2)cc1] | 400 MHz, DMSO-d6 δ 12.13 (s, 1H), 9.06 (d, J = 2.2 Hz, 1H), 8.98 (d, J = 2.0 Hz, 1H), 8.49 (t, J = 2.0 Hz, 1H), 8.39 (s, 1H), 7.78 (d, J = 8.8 Hz, 2H), 7.10 (d, J = 8.8 Hz, 2H), 6.91 (d, J = 2.3 Hz, 2H), 6.60 (t, J = 2.2 Hz, 1H), 4.04 (t, J = 6.5 Hz, 2H), 3.80 (s, 6H), 1.76-1.69 (m, 2H), 1.46 (m, 2H), 0.95 (t, J = 7.4 Hz, 3H). [M + H]+: 434.2. |
| | (E)-5-(4-butoxyphenyl)-N′-(3,5- | |
| | dimethoxybenzylidene)nicotinohydrazide | |
| 28 | 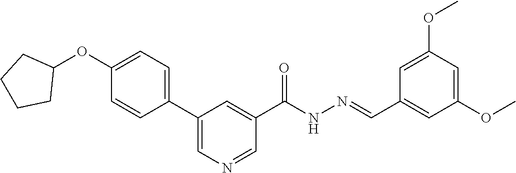 [SMILES: COc1cc(/C=N/NC(=O)c2cncc(-c3ccc(OC4CCCC4)cc3)c2)cc(OC)c1] | 400 MHz, DMSO-d6 δ 12.07 (s, 1H), 9.04 (d, J = 1.9 Hz, 1H), 8.96 (d, J = 1.6 Hz, 1H), 8.44 (sl, 1H), 8.38 (s, 1H), 7.75 (d, J = 8.6 Hz, 2H), 7.06 (d, J = 8.7 Hz, 2H), 6.91 (d, J = 2.1 Hz, 2H), 6.60 (t, J = 1.9 Hz, 1H), 4.89-4.92 (m, 1H), 3.80 (6H), 2.00- 1.89 (m, 2H), 1.79-1.69 (m, 4H), 1.63- 1.56 (m, 2H). [M + H]+: 446.2. |
| | (E)-5-(4-(cyclopentyloxy)phenyl)-N′-(3,5- | |
| | dimethoxybenzylidene)nicotinohydrazide | |
| 29 | 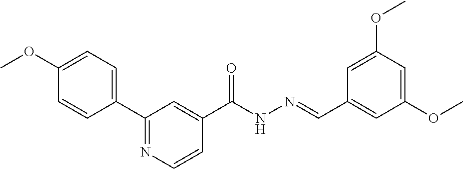 | 400 MHz, DMSO-d6 δ 12.10 (s, 1H), 8.80 (d, J = 5.0 Hz, 1H), 8.41 (s, 1H), 8.27 (s, 1H), 8.11 (d, J = 8 Hz, 2H), 7.69 (dd, J = 5.0, 1.5 Hz, 1H), 7.09 (d, J = 8 Hz, 2H), 6.91 (sinais sobrepostos, 2H), 6.61 (t, J = 2.3 Hz, 1H), 3.84 (s, 3H), 3.81 (s, 6H). [M + H]+: 392.0. |
| | (E)-N′-(3,5-dimethoxybenzylidene)-2-(4- | |
| | methoxyphenyl)isonicotinohydrazide | |
| 30 | 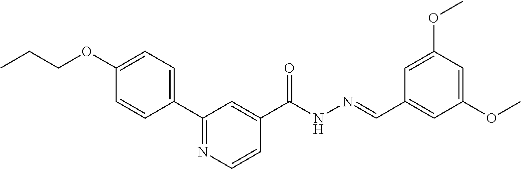 [SMILES: CCCOc1ccc(-c2cc(C(=O)N/N=C/c3cc(OC)cc(OC)c3)ccn2)cc1] | 400 MHz, DMSO-d6 δ 12.10 (s, 1H), 8.79 (d, J = 4.9 Hz, 1H), 8.42 (s, 1H), 8.27 (s, 1H), 8.11 (d, J = 8.6 Hz, 2H), 7.68 (d, J = 4.9 Hz, 1H), 7.08 (d, J = 8.6 Hz, 2H), 6.91 (s, 2H), 6.61 (s, 1H), 4.00 (t, J = 6.5 Hz, 2H), 3.80 (s, 6H), 1.77 (dt, J = 13.9, 7.0 Hz, 2H), 1.00 (t, J = 7.4 Hz, 3H). [M + H]+: 420.3. |
| | (E)-N′-(3,5-dimethoxybenzylidene)-2-(4- | |
| | propoxyphenyl)isonicotinohydrazide | |
| | (Compound 30); | |
| 31 | 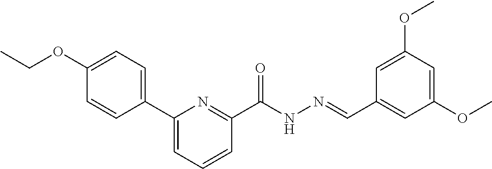 [SMILES: CCOc1ccc(-c2cccc(C(=O)N/N=C/c3cc(OC)cc(OC)c3)n2)cc1] | 400 MHz, DMSO-d6 δ 11.91 (s, 1H), 8.65 (s, 1H), 8.32 (d, J = 8.9 Hz, 2H), 8.16 (dd, J = 8.0, 0.9 Hz, 1H), 8.06 (t, J = 7.8 Hz, 1H), 7.99 (dd, J = 7.6, 0.9 Hz, 1H), 7.07 (d, J = 8.9 Hz, 2H), 6.92 (sinais sobrepostos, 2H), 6.60 (t, J = 2.3 Hz, 1H), 4.12 (q, J = 6.9 Hz, 2H), 3.81 (s, 6H), 1.35 (s, 3H). |
| | (E)-N′-(3,5-dimethoxybenzylidene)-6-(4- | |
| | ethoxyphenyl)picolinohydrazide | |
| | (Compound 31); | |
| 32 | 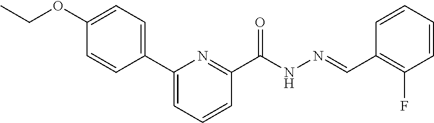 [SMILES: CCOc1ccc(-c2cccc(C(=O)N/N=C/c3ccccc3F)n2)cc1] | 400 MHz, DMSO-d6 δ 12.09 (1H), 9.00 (s, 1H), 8.33 (d, J = 8.8 Hz, 2H), 8.16 (d, J = 7.9 Hz, 1H), 8.06 (t, J = 7.8 Hz, 1H), 8.01 (m, 2H), 7.52 (m, 1H), 7.33 (t, J = 8.6 Hz, 2H), 7.08 (d, J = 8.8 Hz, 2H), 4.12 (q, J = 7.0 Hz, 2H), 1.37 (t, J = 6.9 Hz, 3H). [M + H]+: 364.4. |
| | (E)-6-(4-ethoxyphenyl)-N′-(2- | |
| | fluorobenzylidene)picolinohydrazide | |
| 33 | 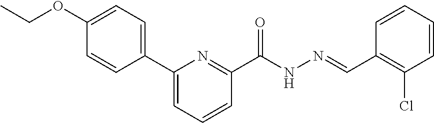 [SMILES: CCOc1ccc(-c2cccc(C(=O)N/N=C/c3ccccc3Cl)n2)cc1] | 400 MHz, DMSO-d6 δ 12.23 (s, 1H), 9.12 (s, 1H), 8.29 (d, J = 8.2 Hz, 2H), 8.13 (d, J = 7.9 Hz, 1H), 8.09-8.00 (m, 2H), 7.99 (d, J = 5 Hz), 7.52 (d, J = 7.5 Hz, 1H), 7.48- 7.40 (m, 2H), 7.05 (d, J = 8.2 Hz, 2H), 4.09 (q, J = 6.8 Hz, 2H), 1.34 (t, J = 6.8 Hz, 3H). [M + H]+: 380.4. |
| | (E)-N′-(2-chlorobenzylidene)-6-(4- | |
| | ethoxyphenyl)picolinohydrazide | |
| 34 | 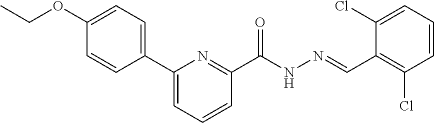 [SMILES: CCOc1ccc(-c2cccc(C(=O)N/N=C/c3c(Cl)cccc3Cl)n2)cc1] | 400 MHz, DMSO-d6 δ 12.26 (s, 1H), 8.92 (s, 1H), 8.32 (d, J = 8.8 Hz, 2H), 8.17 (d, J = 8.0 Hz, 1H), 8.07 (t, J = 7.8 Hz, 1H), 7.99 (d, J = 7.6 Hz, 1H), 7.60 (d, J = 8.2 Hz, 2H), 7.47 (t, J = 8.1 Hz, 1H), 7.08 (d, J = 8.8 Hz, 2H), 4.12 (q, J = Hz, 2H), 1.37 (t, J = 6.9 Hz, 3H). [M + H]+: 414.1. |
| | (E)-N′-(2,6-dichlorobenzylidene)-6-(4- | |
| | ethoxyphenyl)picolinohydrazide | |
| 35 | 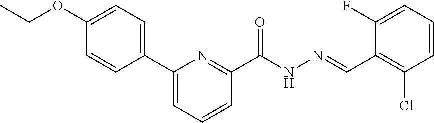 [SMILES: CCOc1ccc(-c2cccc(C(=O)N/N=C/c3c(F)cccc3Cl)n2)cc1] | 400 MHz, DMSO-d6 δ 12.21 (s, 1H), 8.96 (s, 1H), 8.32 (d, J = 8.8 Hz, 2H), 8.17 (d, J = 8.0 Hz, 1H), 8.07 (t, J = 7.8 Hz, 1H), 7.99 (d, J = 7.6 Hz, 1H), 7.55-7.47 (m, 1H), 7.45 (d, J = 7.9 Hz, 1H), 7.40-7.33 (m, 1H), 7.08 (d, J = 8.8 Hz, 2H), 4.12 (q, J = 6.9 Hz, 2H), 1.37 (t, J = 7.0 Hz, 3H). [M + H]+: 398.0. |
| | (E)-N′-(2-chloro-6-fluorobenzylidene)-6- | |
| | (4-ethoxyphenyl)picolinohydrazide | |
| 36 | 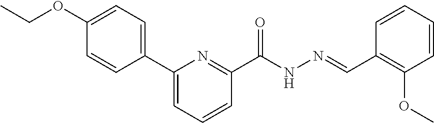 [SMILES: CCOc1ccc(-c2cccc(C(=O)N/N=C/c3ccccc3OC)n2)cc1] | 400 MHz, DMSO-d6 δ 12.00 (s, 1H), 9.04 (s, 1H), 8.33 (d, J = 8.8 Hz, 2H), 8.14 (d, J = 7.9 Hz, 1H), 8.05 (t, J = 7.7 Hz, 1H), 7.98 (d, J = 7.5 Hz, 1H), 7.92 (dd, J = 7.7, 1.6 Hz, 1H) 7.49-7.40 (dt, J = 12 Hz, 4 Hz, 1H), 7.13 (d, J = 8.3 Hz, 1H), 7.06 (dd, J = 14.8, 8.2 Hz, 3H), 4.12 (q, J = 7.0 Hz, 2H), 3.89 (s, 3H), 1.37 (t, J = 7.0 Hz, 3H). [M + H]+: 376.3. |
| | (E)-6-(4-ethoxyphenyl)-N′-(2- | |
| | methoxybenzylidene)picolinohydrazide | |
| 37 | 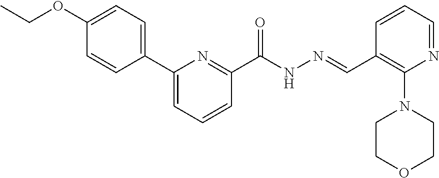 [SMILES: CCOc1ccc(-c2cccc(C(=O)N/N=C/c3cccnc3N3CCOCC3)n2)cc1] | 400 MHz, DMSO-d6 δ 12.09 (s, 1H), 8.77 (s, 1H), 8.33 (dd, J = 4.7, 1.9 Hz, 1H), 8.29 (d, J = 8.8 Hz, 2H), 8.19-8.14 (m, sinais solapados, 2H), 8.06 (t, J = 7.8 Hz, 1H), 7.98 (d, J = 7.1 Hz, 1H), 7.13 (dd, J = 7.6, 4.8 Hz, 1H), 7.08 (d, J 8.8 Hz, 2H), 4.12 (q, J = 7.0 Hz, 2H), 3.85-3.76 (m, 4H), 3.22- 3.14 (m, 4H), 1.37 (t, J = 7.0 Hz, 3H). [M + H]+: 431.9. |
| | (E)-6-(4-ethoxyphenyl)-N′-((2- | |
| | morpholinopyridin-3- | |
| | yl)methylene)picolinohydrazide | |
| 38 | 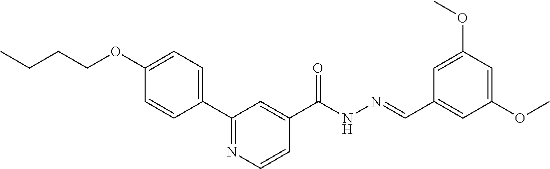 [SMILES: CCCCOc1ccc(-c2cc(C(=O)N/N=C/c3cc(OC)cc(OC)c3)ccn2)cc1] | 400 MHz, DMSO-d6 δ 12.10 (s, 1H), 8.79 (d, J = 5.4 Hz, 1H), 8.41 (s, 1H), 8.27 (s, 1H), 8.11 (d, J = 6.9 Hz, 2H), 7.68 (dd, J = 5.0, 1.5 Hz, 1H), 7.08 (d, J = 8.9 Hz, 2H), 6.91(sinais sobrepostos, 2H), 6.61 (t, J = 2.3 Hz, 1H), 4.05 (t, J = 6.5 Hz, 2H), 3.81 (s, 6H), 1.73 (dt, J = 14.3, 6.5 Hz, 2H), 1.47 (dt, J = 14.9, 7.4 Hz, 2H), 0.95 (t, J = 7.4 Hz, 3H). [M + H]+: 434.2. |
| | (E)-2-(4-butoxyphenyl)-N′-(3,5- | |
| | dimethoxybenzylidene)isonicotinohydrazide | |
| 39 | 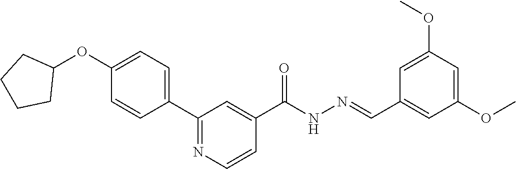 [SMILES: COc1cc(/C=N/NC(=O)c2ccnc(-c3ccc(OC4CCCC4)cc3)c2)cc(OC)c1] | 400 MHz, DMSO-d6 δ 12.10 (s, 1H), 8.79 (d, J = 5.0 Hz, 1H), 8.41 (s, 1H), 8.26 (s, 1H), 8.09 (d, J = 8.8 Hz, 2H), 7.68 (dd, J = 5.0, 1.3 Hz, 1H), 7.05 (d, J = 8.8 Hz, 2H), 6.91 (sinais sobrepostos, 2H), 6.61 (t, J = 2.2 Hz, 1H), 4.91 (s, 1H), 3.81 (s, 6H), 1.95 (m, 2H), 1.74 (m, 4H), 1.61 (m, 2H). [M + H]+: 445.7. |
| | (E)-2-(4-(cyclopentyloxy)phenyl)-N′-(3,5- | |
| | dimethoxybenzylidene)isonicotinohydrazide | |
| 40 | 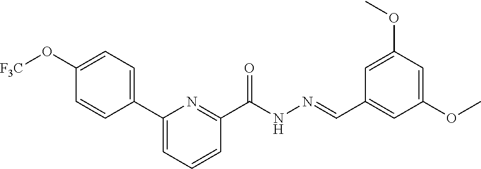 [SMILES: COc1cc(/C=N/NC(=O)c2cccc(-c3ccc(OC(F)(F)F)cc3)n2)cc(OC)c1] | 400 MHz, DMSO-d6 δ 11.95 (s, 1H), 8.65 (s, 1H), 8.49 (d, J = 8.9 Hz, 2H), 8.27 (dd, J = 7.9, 1.0 Hz, 1H), 8.16 (t, J = 7.8 Hz, 1H), 8.10 (dd, J = 7.7, 1.0 Hz, 1H), 7.53 (d, J = 8.1 Hz, 2H), 6.92 (overlapping signs, 2H), 6.60 (t, J = 2.3 Hz, 1H), 3.81 (s, 6H). [M + H]+: 445.7. |
| | (E)-N′-(3,5-dimethoxybenzylidene)-6-(4- | |
| | (trifluoromethoxy)phenyl)picolinohydrazide | |
| 41 | 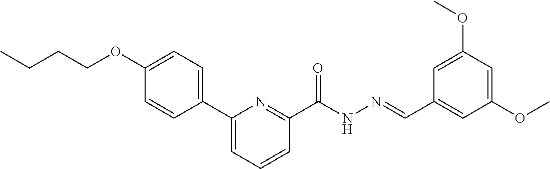 [SMILES: CCCCOc1ccc(-c2cccc(C(=O)N/N=C/c3cc(OC)cc(OC)c3)n2)cc1] | 400 MHz, DMSO-d6 δ 11.90 (s, 1H), 8.66 (s, 1H), 8.30 (d, J = 8.9 Hz, 2H), 8.14 (d, J = 7.9 Hz, 1H), 8.05 (H-3, t, J = 7.8 Hz, 1H), 7.99 (d, J = 8.3 Hz, 1H), 7.06 (d, J = 8.9 Hz, 2H), 6.92 (sinais sobrepostos, 2H), 6.60 (t, J = 2.3 Hz, 1H), 4.05 (t, J = 6.5 Hz, 2H), 3.79 (s, 6H), 1.73 (dt, J = 14.3, 6.5 Hz, 2H), 1.46 (h, J = 7.4 Hz, 2H), 0.95 (t, J = 7.4 Hz, 3H). [M + H]+: 434.3. |
| | (E)-6-(4-butoxyphenyl)-N′-(3,5- | |
| | dimethoxybenzylidene)picolinohydrazide | |
| 42 | 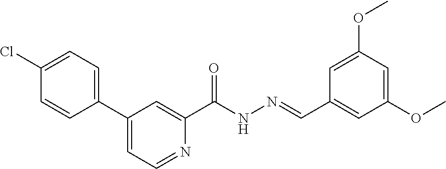 [SMILES: COc1cc(/C=N/NC(=O)c2cc(-c3ccc(Cl)cc3)ccn2)cc(OC)c1] | 400 MHz, DMSO-d6 δ 12.19 (s, 1H), 8.77 (d, J = 5.3 Hz, 1H), 8.57 (s, 1H), 8.34 (s, 1H), 7.99 (dd, J = 1.8, 5.1 Hz, 1H), 7.92 (d, J = 8.6 Hz, 2H), 7.62 (d, J = 8.6 Hz, 2H), 6.85 (d, J = 2.0 Hz, 2H), 6.58 (s, 1H), 3.79 (s, 6H). [M + H]+: 396.1. |
| | (E)-2-(4-chlorophenyl)-N′-(3,5- | |
| | dimethoxybenzylidene)isonicotinohydrazide | |
| 43 | 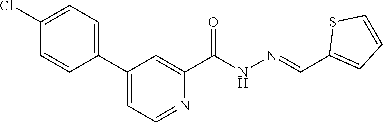 [SMILES: O=C(N/N=C/c1cccs1)c1cc(-c2ccc(Cl)cc2)ccn1] | 400 MHz, DMSO-d6 δ 12.20 (s, 1H), 8.84 (s, 1H), 8.76 (d, J = 5.1 Hz, 1H), 8.33 (s, 1H), 7.98 (dd, J = 1.7, 5.0 Hz, 1H), 7.92 (d, J = 8.4 Hz, 2H), 7.68 (d, J = 4.9 Hz, 1H), 7.61 (d, J = 8.6 Hz, 2H), 7.42 (d, J = 3.1 Hz, 1H), 7.19-7.08 (m, 1H). [M + H]+: 342.0. |
| | (E)-6-(4-chlorophenyl)-N′-(thiophen-2- | |
| | ylmethylene)picolinohydrazide | |
| 44 | 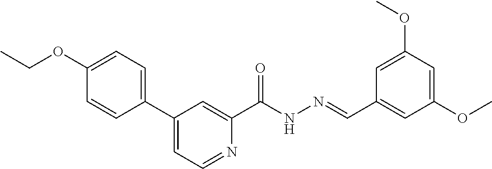 [SMILES: CCOc1ccc(-c2ccnc(C(=O)N/N=C/c3cc(OC)cc(OC)c3)c2)cc1] | 400 MHz, DMSO-d6 δ 12.18 (s, 1H), 8.71 (d, J = 5.3 Hz, 1H), 8.59 (s, 1H), 8.32 (d, J = 1.2 Hz, 1H), 7.95 (dd, J = 1.9, 5.2 Hz, 1H), 7.86 (d, J = 8.8 Hz, 2H), 7.11 (d, J = 8.8 Hz, 2H), 6.87 (d, J = 2.2 Hz, 2H), 6.60 (t, J = 2.3 Hz, 1H), 4.12 (q, J = 7.0 Hz, 2H), 3.81 (s, 6H), 1.37 (t, J = 7.0 Hz, 3H). [M + H]+: 406.1. |
| | (E)-N′-(3,5-dimethoxybenzylidene)-6-(4- | |
| | ethoxyphenyl)picolinohydrazide | |
| 45 | 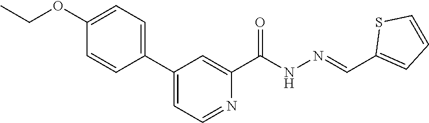 [SMILES: CCOc1ccc(-c2ccnc(C(=O)N/N=C/c3cccs3)c2)cc1] | 400 MHz, DMSO-d6 δ 12.18 (s, 1H), 8.85 (s, 1H), 8.70 (d, J = 5.3 Hz, 1H), 8.30 (d, J = 1.3 Hz, 1H), 7.94 (dd, J = 2.0, 5.1 Hz, 1H), 7.85 (d, J = 8.8 Hz, 2H), 7.69 (d, J = 5.0 Hz, 1H), 7.48-7.41 (m, 1H), 7.21- 7.06 (m, 3H), 4.11 (q, J = 7.0 Hz, 2H), 1.36 (t, J = 7.0 Hz, 3H). [M + H]+: 352.0. |
| | (E)-6-(4-ethoxyphenyl)-N′-(thiophen-2- | |
| | ylmethylene)picolinohydrazide | |
| 46 | 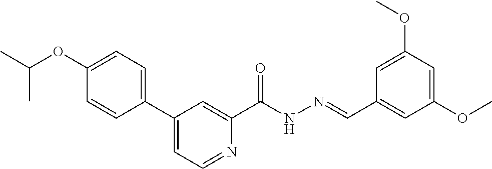 [SMILES: COc1cc(/C=N/NC(=O)c2cc(-c3ccc(OC(C)C)cc3)ccn2)cc(OC)c1] | 400 MHz, DMSO-d6 δ 2.19 (s, 1H), 8.71 (d, J = 5.3 Hz, 1H), 8.58 (s, 1H), 8.31 (s, 1H), 7.95 (dd, J = 1.8, 5.3 Hz, 1H), 7.84 (d, J = 8.8 Hz, 2H), 7.09 (d, J = 8.8 Hz, 2H), 6.86 (d, J = 2.0 Hz, 2H), 6.60-6.58 (m, 1H), 4.73 (td, J = 6.0, 12.1 Hz, 1H), 3.81 (s, 6H), 1.31 (d, J = 6.0 Hz, 6H). [M + H]+: 420.1. |
| | (E)-N′-(3,5-dimethoxybenzylidene)-6-(4- | |
| | isopropoxyphenyl)picolinohydrazide | |
| 47 | 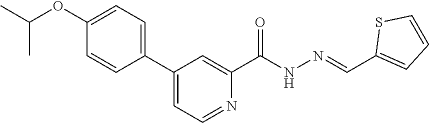 [SMILES: CC(C)Oc1ccc(-c2ccnc(C(=O)N/N=C/c3cccs3)c2)cc1] | 400 MHz, DMSO-d6 δ 12.20 (s, 1H), 8.85 (s, 1H), 8.70 (d, J = 5.1 Hz, 1H), 8.30 (d, J = 1.3 Hz, 1H), 7.94 (dd, J = 1.9, 5.2 Hz, 1H), 7.84 (d, J = 8.8 Hz, 2H), 7.70 (d, J = 5.1 Hz, 1H), 7.44 (d, J = 2.9 Hz, 1H), 7.16 (dd, J = 3.7, 4.9 Hz, 1H), 7.09 (d, J = 8.6 Hz, 2H), 4.79-4.66 (m, 1H), 1.31 (d, J = 6.0 Hz, 6H). [M + H]+: 366.1. |
| | (E)-4-(4-isopropoxyphenyl)-N′-(thiophen- | |
| | 2-ylmethylene)picolinohydrazide | |
| 48 | 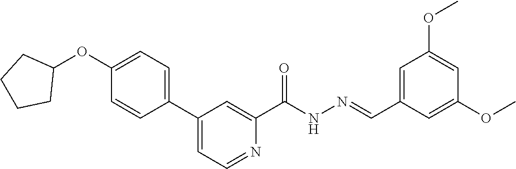 [SMILES: COc1cc(/C=N/NC(=O)c2cc(-c3ccc(OC4CCCC4)cc3)ccn2)cc(OC)c1] | 400 MHz, CDCl3 δ 11.09-11.01 (m, 1H), 8.61-8.49 (m, 2H), 8.31-8.22 (m, 1H), 7.72-7.62 (m, 3H), 6.98 (s, 4H), 6.57- 6.48 (m, 1H), 4.87-4.80 (m, 1H), 3.85 (s, 6H), 2.02-1.75 (m, 6H), 1.72-1.61 (m, 2H). [M + H]+: 446.1. |
| | (E)-4-(4-(cyclopentyloxy)phenyl)-N′- | |
| | (3,5- | |
| | dimethoxybenzylidene)picolinohydrazide | |
| 49 | 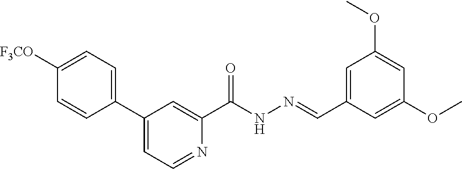 [SMILES: COc1cc(/C=N/NC(=O)c2cc(-c3ccc(OC(F)(F)F)cc3)ccn2)cc(OC)c1] | 400 MHz, CDCl3 δ 10.95 (s, 1H), 8.57 (d, J = 5.1 Hz, 1H), 8.46 (d, J = 1.2 Hz, 1H), 8.19 (s, 1H), 7.70 (d, J = 7.4 Hz, 2H), 7.61 (dd, J = 1.9, 5.1 Hz, 1H), 7.30 (d, J = 8.1 Hz, 2H), 7.19 (s, 1H), 6.91 (d, J = 2.3 Hz, 2H), 6.46 (t, J = 2.3 Hz, 1H), 3.78 (s, 6H). [M + H]+: 446.1. |
| | (E)-N′-(3,5-dimethoxybenzylidene)-6-(4- | |
| | (trifluoromethoxy)phenyl)picolinohydrazide | |
| 50 | 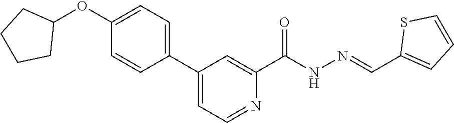 [SMILES: O=C(N/N=C/c1cccs1)c1cc(-c2ccc(OC3CCCC3)cc2)ccn1] | 400 MHz, CDCl3 δ 11.02-10.94 (m, 1H), 8.76-8.69 (m, 1H), 8.57-8.53 (m, 1H), 8.50-8.47 (m, 1H), 7.72-7.62 (m, 3H), 7.47-7.41 (m, 1H), 7.38-7.35 (m, 1H), 7.11-7.05 (m, 1H), 7.02-6.96 (m, 2H), 4.88-4.79 (m, 1H), 2.02-1.76 (m, 6H), 1.72-1.61 (m, 2H). [M + H]+: 392.1. |
| | (E)-4-(4-(cyclopentyloxy)phenyl)-N′- | |
| | (thiophen-2- | |
| | ylmethylene)picolinohydrazide | |
| 51 | 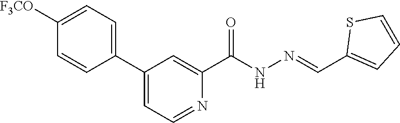 [SMILES: O=C(N/N=C/c1cccs1)c1cc(-c2ccc(OC(F)(F)F)cc2)ccn1] | 400 MHz, CDCl3 δ 10.88 (s, 1H), 8.65 (s, 1H), 8.56 (d, J = 5.1 Hz, 1H), 8.43 (d, J = 1.2 Hz, 1H), 7.69 (d, J = 7.4 Hz, 2H), 7.59 (dd, J = 1.8, 5.0 Hz, 1H), 7.38 (d, J = 5.0 Hz, 1H), 7.33-7.28 (m, 3H), 7.19 (s, 1H), 7.02 (dd, J = 3.7, 5.0 Hz, 1H). [M + H]+: 392.1. |
| | (E)-N′-(thiophen-2-ylmethylene)-4-(4- | |
| | (trifluoromethoxy)phenyl)picolinohydrazide | |
| 52 | 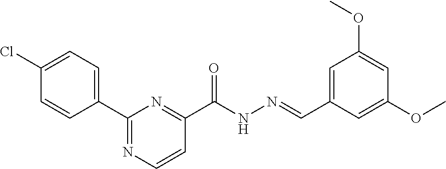 [SMILES: COc1cc(/C=N/NC(=O)c2ccnc(-c3ccc(Cl)cc3)n2)cc(OC)c1] | 400 MHz, DMSO-d6 δ 3.74-3.85 (m, 6 H) 6.61 (t, J = 2.20 Hz, 1 H) 6.92 (d, J = 2.21 Hz, 2 H) 7.64 (d, J = 8.60 Hz, 2 H) 8.00 (d, J = 4.85 Hz, 1 H) 8.63-8.73 (m, 3 H) 9.18 (d, J = 4.85 Hz, 1 H) 12.20 (s, 1 H). [M + H]+: 397.2. |
| | (E)-2-(4-chlorophenyl)-N′-(3,5- | |
| | dimethoxybenzylidene)pyrimidine-4- | |
| | carbohydrazide | |
| 53 | 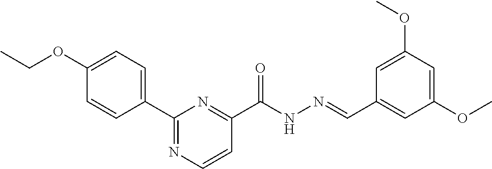 [SMILES: CCOc1ccc(-c2nccc(C(=O)N/N=C/c3cc(OC)cc(OC)c3)n2)cc1] | 400 MHz, CDCl3 δ 10.92 (s, 1H), 9.02 (d, J = 4.8 Hz, 1H), 8.44-8.39 (m, 3H), 8.00 (d, J = 4.8 Hz, 1H), 7.05-6.98 (m, 4H), 6.55 (s, 1H), 4.14 (q, J = 6.7 Hz, 2H), 3.85 (s, 6H), 1.47 (t, J = 6.8 Hz, 3H). [M + H]+: 407.1. |
| | (E)-N′-(3,5-dimethoxybenzylidene)-2-(4- | |
| | ethoxyphenyl)pyrimidine-4- | |
| | carbohydrazide | |
| 54 | 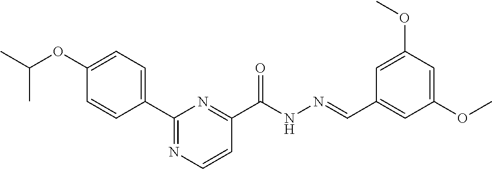 [SMILES: COc1cc(/C=N/NC(=O)c2ccnc(-c3ccc(OC(C)C)cc3)n2)cc(OC)c1] | 400 MHz, DMSO-d6 δ 12.14 (s, 1H), 12.18- 12.10 (m, 1H), 9.11 (d, J = 4.9 Hz, 1H), 8.70 (s, 1H), 8.61 (d, J = 8.8 Hz, 2H), 7.90 (d, J = 4.9 Hz, 1H), 7.09 (d, J = 8.9 Hz, 2H), 6.94 (d, J = 2.3 Hz, 2H), 6.63 (s, 1H), 4.84-4.73 (m, 1H), 3.82 (s, 6H), 1.33 (d, J = 6.0 Hz, 6H). [M + H]+: 421.1. |
| | (E)-N′-(3,5-dimethoxybenzylidene)-2-(4- | |
| | isopropoxyphenyl)pyrimidine-4- | |
| | carbohydrazide | |
| 55 | 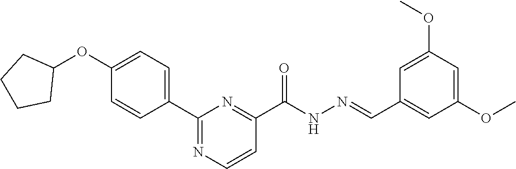 [SMILES: COc1cc(/C=N/NC(=O)c2ccnc(-c3ccc(OC4CCCC4)cc3)n2)cc(OC)c1] | 400 MHz, DMSO-d6 δ 12.14 (s, 1H), 9.11 (d, J = 4.9 Hz, 1H), 8.70 (s, 1H), 8.61 (d, J = 8.8 Hz, 2H), 7.90 (d, J = 4.9 Hz, 1H), 7.07 (d, J = 8.8 Hz, 2H), 6.94 (d, J = 2.1 Hz, 2H), 6.63 (s, 1H), 5.01-4.91 (m, 1H), 3.82 (s, 6H), 1.99 (br t, J = 5.6 Hz, 2H), 1.81-1.68 (m, 4H), 1.62 (br s, 2H). [M + H]+: 447.1. |
| | (E)-2-(4-(cyclopentyloxy)phenyl)-N′- | |
| | (3,5-dimethoxybenzylidene)pyrimidine-4- | |
| | carbohydrazide | |
| 56 | 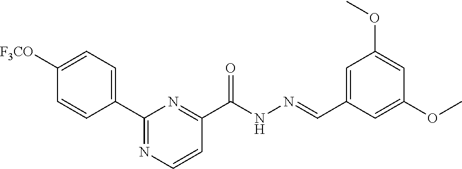 [SMILES: COc1cc(/C=N/NC(=O)c2ccnc(-c3ccc(OC(F)(F)F)cc3)n2)cc(OC)c1] | 400 MHz, DMSO-d6 δ 12.21 (s, 1H), 9.22 (d, J = 4.9 Hz, 1H), 8.80 (d, J = 8.0 Hz, 2H), 8.70 (s, 1H), 8.04 (d, J = 5.0 Hz, 1H), 7.58 (d, J = 8.1 Hz, 2H), 6.94 (d, J = 2.2 Hz, 2H), 6.63 (t, J = 2.3 Hz, 1H), 3.82 (s, 6H). [M + H]+: 447.1. |
| | (E)-N′-(3,5-dimethoxybenzylidene)-2- | |
| | (4-(trifluoromethoxy)phenyl)pyrimidine-4- | |
| | carbohydrazide | |
| 57 | 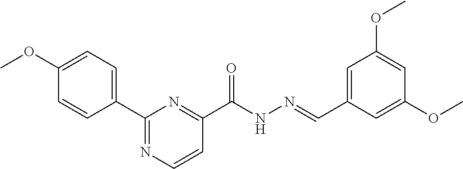 | 400 MHz, DMSO-d6 δ 10.97-10.86 (m, 1H), 9.06-8.95 (m, 1H), 8.47-8.37 (m, 3H), 8.04-7.94 (m, 1H), 7.08-7.02 (m, 2H), 7.00-6.96 (m, 2H), 6.59-6.49 (m, 1H), 3.91 (s, 3H), 3.87-3.82 (m, 6H). [M + H]+: 393.6. |
| | (E)-N′-(3,5-dimethoxybenzylidene)-2-(4- | |
| | methoxyphenyl)pyrimidine-4- | |
| | carbohydrazide | |
| 58 | 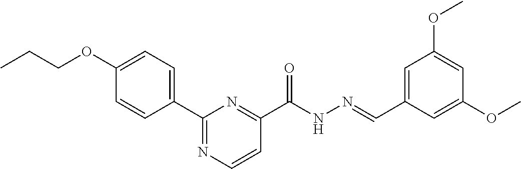 [SMILES: CCCOc1ccc(-c2nccc(C(=O)N/N=C/c3cc(OC)cc(OC)c3)n2)cc1] | 400 MHz, DMSO-d6 δ 10.95 (s, 1H), 9.04 (d, J = 4.9 Hz, 1H), 8.48-8.41 (m, 3H), 8.03 (d, J = 4.8 Hz, 1H), 7.09-6.99 (m, 4H), 6.58 (t, J = 2.1 Hz, 1H), 4.05 (t, J = 6.5 Hz, 2H), 3.88 (s, 6H), 1.89 (sxt, J = 7.1 Hz, 2H), 1.11 (t, J = 7.5 Hz, 3H). [M + H]+: 421.1. |
| | (E)-N′-(3,5-dimethoxybenzylidene)-2-(4- | |
| | isopropoxyphenyl)pyrimidine-4- | |
| | carbohydrazide | |
| 59 | 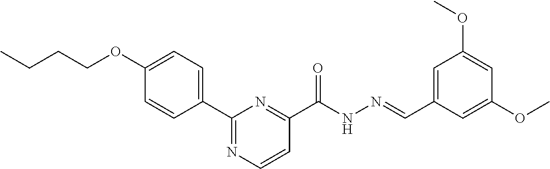 [SMILES: CCCCOc1ccc(-c2nccc(C(=O)N/N=C/c3cc(OC)cc(OC)c3)n2)cc1] | 400 MHz, DMSO-d6 δ 12.14 (s, 1H), 9.12 (d, J = 5.0 Hz, 1H), 8.70 (s, 1H), 8.65-8.59 (m, J = 8.9 Hz, 2H), 7.91 (d, J = 5.0 Hz, 1H), 7.14-7.07 (m, J = 8.9 Hz, 2H), 6.94 (d, J = 2.3 Hz, 2H), 6.63 (t, J = 2.3 Hz, 1H), 4.10 (t, J = 6.5 Hz, 2H), 3.82 (s, 6H), 1.80- 1.71 (m, 2H), 1.48 (sxt, J = 7.5 Hz, 2H), 0.97 (t, J = 7.4 Hz, 3H). [M + H]+: 435.1. |
| | (E)-2-(4-butoxyphenyl)-N′-(3,5- | |
| | dimethoxybenzylidene)pyrimidine-4- | |
| | carbohydrazide | |
| 60 | 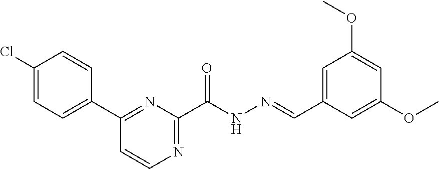 [SMILES: COc1cc(/C=N/NC(=O)c2nccc(-c3ccc(Cl)cc3)n2)cc(OC)c1] | 400 MHz, DMSO-d6 δ 11.90 (s, 1H), 9.03 (br d, J = 4.6 Hz, 1H), 8.58 (br s, 1H), 8.36 (br d, J = 7.9 Hz, 2H), 8.21 (br d, J = 4.2 Hz, 1H), 7.63 (br d, J = 8.4 Hz, 2H), 6.90 (br s, 2H), 6.58 (br s, 1H), 3.81 (br s, 6H). [M + H]+: 397.1. |
| | (E)-4-(4-chlorophenyl)-N′-(3,5- | |
| | dimethoxybenzylidene)pyrimidine-2- | |
| | carbohydrazide | |
| 61 | 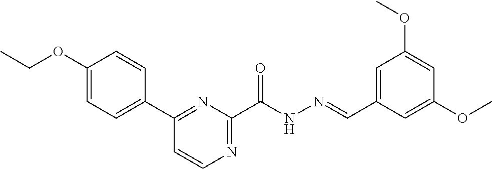 [SMILES: CCOc1ccc(-c2ccnc(C(=O)N/N=C/c3cc(OC)cc(OC)c3)n2)cc1] | 400 MHz, DMSO-d6 δ 11.88 (br s, 1H), 8.91 (br d, J = 4.9 Hz, 1H), 8.57 (br s, 1H), 8.30 (br d, J = 8.2 Hz, 2H), 8.11 (br d, J = 4.9 Hz, 1H), 7.10 (br d, J = 7.9 Hz, 2H), 6.90 (br s, 2H), 6.58 (br s, 1H), 4.16 (br d, J = 6.0 Hz, 2H), 3.81 (br s, 6H), 1.37 (br t, J = 6.5 Hz, 3H). [M + H]+: 407.1. |
| | (E)-N′-(3,5-dimethoxybenzylidene)-4-(4- | |
| | ethoxyphenyl)pyrimidine-2- | |
| | carbohydrazide | |
| 62 | 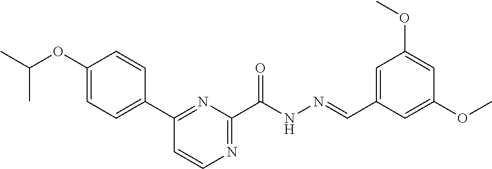 [SMILES: COc1cc(/C=N/NC(=O)c2nccc(-c3ccc(OC(C)C)cc3)n2)cc(OC)c1] | 400 MHz, DMSO-d6 δ 11.85 (s, 1H), 8.91 (br d, J = 5.3 Hz, 1H), 8.57 (br s, 1H), 8.28 (br d, J = 7.7 Hz, 2H), 8.09 (br d, J = 4.2 Hz, 1H), 7.08 (br d, J = 8.4 Hz, 2H), 6.90 (br s, 2H), 6.58 (br s, 1H), 4.74 (td, J = 6.0, 11.8 Hz, 1H), 3.81 (br s, 6H), 1.32 (d, J = 6.0 Hz, 6H). [M + H]+: 421.2. |
| | (E)-N′-(3,5-dimethoxybenzylidene)-4-(4- | |
| | isopropoxyphenyl)pyrimidine-2- | |
| | carbohydrazide | |
| 63 | 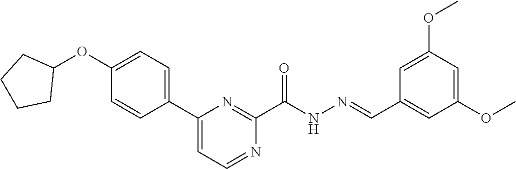 [SMILES: COc1cc(/C=N/NC(=O)c2nccc(-c3ccc(OC4CCCC4)cc3)n2)cc(OC)c1] | 400 MHz, DMSO-d6 δ 12.15 (s, 1H), 8.96 (d, J = 5.5 Hz, 1H), 8.58 (s, 1H), 8.36 (d, J = 8.9 Hz, 2H), 8.19 (d, J = 5.5 Hz, 1H), 7.10 (d, J = 8.9 Hz, 2H), 6.91 (d, J = 2.2 Hz, 2H), 6.61 (t, J = 2.3 Hz, 1H), 4.97 (br t, J = 5.9 Hz, 1H), 3.82 (s, 6H), 2.03-1.93 (m, 2H), 1.79-1.69 (m, 4H), 1.67-1.57 (m, 2H). [M + H]+: 447.1. |
| | (E)-4-(4-(cyclopentyloxy)phenyl)-N′- | |
| | (3,5-dimethoxybenzylidene)pyrimidine-2- | |
| | carbohydrazide | |
| 64 | 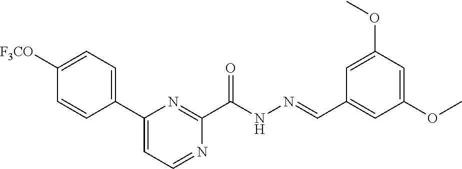 [SMILES: COc1cc(/C=N/NC(=O)c2nccc(-c3ccc(OC(F)(F)F)cc3)n2)cc(OC)c1] | 400 MHz, DMSO-d6 δ 11.94 (s, 1H), 9.07 (br d, J = 4.8 Hz, 1H), 8.60 (br s, 1H), 8.49 (br d, J = 7.1 Hz, 2H), 8.26 (br d, J = 3.8 Hz, 1H), 7.57 (br d, J = 8.2 Hz, 2H), 6.93 (br s, 2H), 6.61 (br s, 1H), 3.83 (br s, 6H). [M + H]+: 447.1. |
| | (E)-N′-(3,5-dimethoxybenzylidene)-4-(4- | |
| | (trifluoromethoxy)phenyl)pyrimidine-2- | |
| | carbohydrazide | |
| 65 | 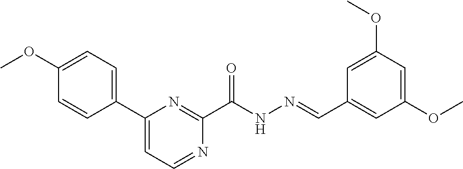 | 400 MHz, DMSO-d6 δ 11.86 (br s, 1H), 8.92 (br d, J = 4.6 Hz, 1H), 8.57 (br s, 1H), 8.31 (br d, J = 7.1 Hz, 2H), 8.11 (br d, J = 4.4 Hz, 1H), 7.12 (br d, J = 8.4 Hz, 2H), 6.90 (br s, 2H), 6.58 (br s, 1H), 3.87 (s, 3H), 3.81 (br s, 6H). [M + H]+: 393.1. |
| | (E)-N′-(3,5-dimethoxybenzylidene)-4-(4- | |
| | methoxyphenyl)pyrimidine-2- | |
| | carbohydrazide | |
| 66 | 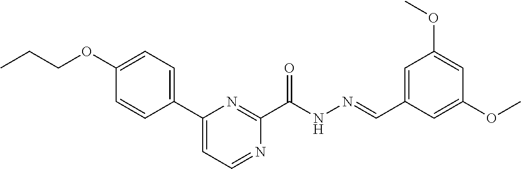 [SMILES: CCCOc1ccc(-c2ccnc(C(=O)N/N=C/c3cc(OC)cc(OC)c3)n2)cc1] | 400 MHz, DMSO-d6 δ 11.87 (s, 1H), 8.94 (br d, J = 5.1 Hz, 1H), 8.60 (br s, 1H), 8.32 (br d, J = 7.8 Hz, 2H), 8.12 (br d, J = 4.5 Hz, 1H), 7.13 (br d, J = 8.7 Hz, 2H), 6.93 (br s, 2H), 6.61 (br s, 1H), 4.08 (t, J = 6.5 Hz, 2H), 3.83 (br s, 6H), 1.80 (sxt, J = 7.1 Hz, 2H), 1.03 (t, J = 7.4 Hz, 3H). [M + H]+: 421.1. |
| | (E)-N′-(3,5-dimethoxybenzylidene)-4-(4- | |
| | propoxyphenyl)pyrimidine-2- | |
| | carbohydrazide | |
| 67 | 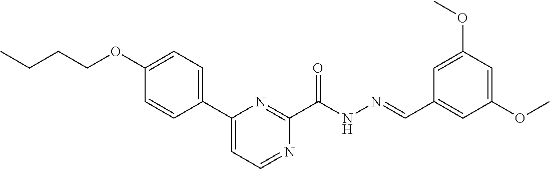 [SMILES: CCCCOc1ccc(-c2ccnc(C(=O)N/N=C/c3cc(OC)cc(OC)c3)n2)cc1] | 400 MHz, DMSO-d6 δ 11.85 (s, 1H), 8.91 (br d, J = 4.9 Hz, 1H), 8.57 (br s, 1H), 8.29 (br d, J = 7.5 Hz, 2H), 8.09 (br d, J = 4.0 Hz, 1H), 7.10 (br d, J = 8.6 Hz, 2H), 6.90 (br s, 2H), 6.58 (br s, 1H), 4.10 (t, J = 6.4 Hz, 2H), 3.81 (br s, 6H), 1.80-1.69 (m, 2H), 1.47 (sxt, J = 7.3 Hz, 2H), 0.95 (t, J = 7.4 Hz, 3H). [M + H]+: 435.2. |
| | (E)-4-(4-butoxyphenyl)-N′-(3,5- | |
| | dimethoxybenzylidene)pyrimidine-2- | |
| | carbohydrazide | |
| 68 | 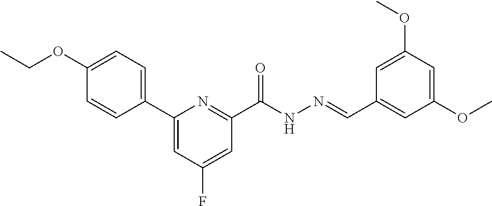 [SMILES: CCOc1ccc(-c2cc(F)cc(C(=O)N/N=C/c3cc(OC)cc(OC)c3)n2)cc1] | 400 MHz, DMSO-d6 δ 11.96 (s, 1H), 8.67 (s, 1H), 8.37 (d, J = 8.9 Hz, 2H), 8.13 (dd, J = 2.1, 10.6 Hz, 1H), 7.78 (dd, J = 2.1, 8.8 Hz, 1H), 7.08 (d, J = 8.8 Hz, 2H), 6.93 (d, J = 2.1 Hz, 2H), 6.62 (t, J = 2.2 Hz, 1H), 4.14 (q, J = 7.0 Hz, 2H), 3.82 (s, 6H), 1.38 (t, J = 6.9 Hz, 3H). [M + H]+: 424.0. |
| | (E)-N′-(3,5-dimethoxybenzylidene)-6-(4- | |
| | ethoxyphenyl)-4-fluoropicolinohydrazide | |
| 69 | 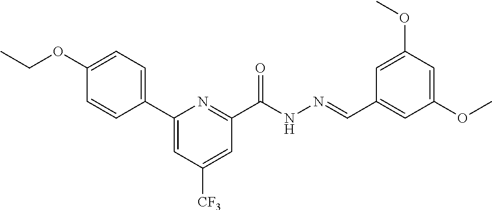 [SMILES: CCOc1ccc(-c2cc(C(F)(F)F)cc(C(=O)N/N=C/c3cc(OC)cc(OC)c3)n2)cc1] | 400 MHz, DMSO-d6 δ 12.05 (s, 1H), 8.66 (s, 1H), 8.51-8.40 (m, 3H), 8.13 (s, 1H), 7.08 (d, J = 9.0 Hz, 2H), 6.91 (d, J = 2.2 Hz, 2H), 6.59 (t, J = 2.2 Hz, 1H), 4.13 (q, J = 7.0 Hz, 2H), 3.79 (s, 6H), 1.36 (t, J = 7.0 Hz, 3H). [M + H]+: 474.1. |
| | (E)-N′-(3,5-dimethoxybenzylidene)-6-(4- | |
| | ethoxyphenyl)picolinohydrazide | |
| 70 | 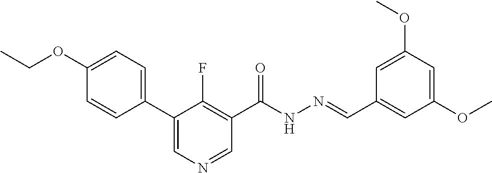 [SMILES: CCOc1ccc(-c2cncc(C(=O)N/N=C/c3cc(OC)cc(OC)c3)c2F)cc1] | |
| | (E)-N′-(3,5-dimethoxybenzylidene)-5-(4-ethoxyphenyl)-4-fluoronicotinohydrazide | |
| 71 | 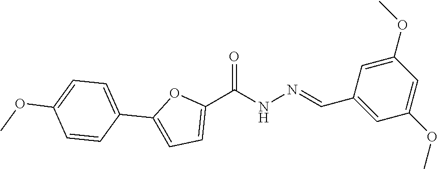 [SMILES: COc1ccc(-c2ccc(C(=O)N/N=C/c3cc(OC)cc(OC)c3)o2)cc1] | 400 MHz, DMSO-d6 δ 11.77 (s, 1H), 8.44 (br s, 1H), 7.89 (br d, J = 6.7 Hz, 2H), 7.37 (br s, 1H), 7.12-7.00 (m, 3H), 6.89 (d, J = 1.8 Hz, 2H), 6.59 (t, J = 1.9 Hz, 1H), 3.82- 3.80 (m, 1H), 3.91-3.74 (m, 8H). [M + H]+: 381.1. |
| | (E)-N′-(3,5-dimethoxybenzylidene)-5-(4- | |
| | methoxyphenyl)furan-2-carbohydrazide | |
| 72 | 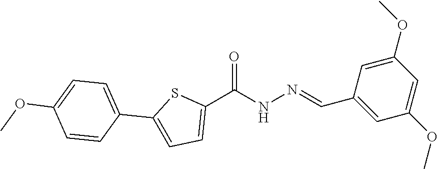 [SMILES: COc1ccc(-c2ccc(C(=O)N/N=C/c3cc(OC)cc(OC)c3)s2)cc1] | 400 MHz, DMSO-d6 δ 11.90 (br s, 1H), 8.50-7.82 (m, 2H), 7.68 (br d, J = 8.6 Hz, 2H), 7.49 (br s, 1H), 7.04 (br d, J = 8.6 Hz, 3H), 6.89 (br s, 1H), 6.58 (t, J = 2.0 Hz, 1H), 3.82 (s, 9H). [M + H]+: 397.1. |
| | (E)-N′-(3,5-dimethoxybenzylidene)-5-(4- | |
| | methoxyphenyl)thiophene-2- | |
| | carbohydrazide | |
| 73 | 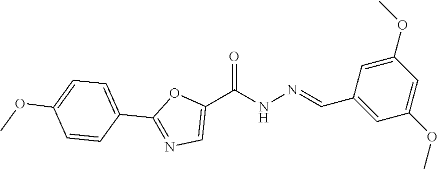 [SMILES: COc1ccc(-c2ncc(C(=O)N/N=C/c3cc(OC)cc(OC)c3)o2)cc1] | 400 MHz, DMSO-d6 δ 12.10-11.88 (m, 1H), 8.45-7.99 (m, 4H), 7.16 (d, J = 8.7 Hz, 2H), 6.89 (br s, 2H), 6.61 (t, J = 2.1 Hz, 1H), 3.87 (s, 3H), 3.81 (s, 6H). [M + H]+: 382.0. |
| | (E)-N′-(3,5-dimethoxybenzylidene)-2-(4- | |
| | methoxyphenyl)oxazole-5- | |
| | carbohydrazide | |
| 74 | 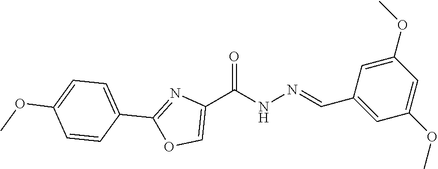 [SMILES: COc1ccc(-c2nc(C(=O)N/N=C/c3cc(OC)cc(OC)c3)co2)cc1] | 400 MHz, DMSO-d6 δ 9.93 (s, 1H), 8.28 (s, 1H), 8.16 (s, 1H), 7.94 (d, J = 8.4 Hz, 2H), 6.94 (br d, J = 8.4 Hz, 2H), 6.89 (d, J = 2.0 Hz, 2H), 6.46 (s, 1H), 3.83-3.81 (m, 3H), 3.78 (s, 6H). [M + H]+: 382.1. |
| | (E)-N′-(3,5-dimethoxybenzylidene)-2-(4- | |
| | methoxyphenol)oxazole-4- | |
| | carbohydrazide | |
| 75 | 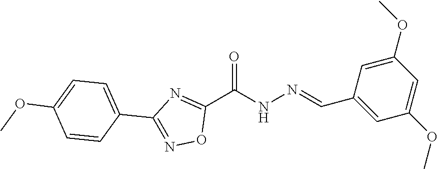 [SMILES: COc1ccc(-c2noc(C(=O)N/N=C/c3cc(OC)cc(OC)c3)n2)cc1] | 400 MHz, DMSO-d6 δ 10.13 (s, 1H), 8.35 (s, 1H), 8.06 (d, J = 8.8 Hz, 2H), 7.03 (d, J = 8.8 Hz, 2H), 6.96 (d, J = 2.2 Hz, 2H), 6.58 (t, J = 2.1 Hz, 1H), 3.90 (s, 3H), 3.86 (s, 6H). [M + H]+: 383.1. |
| | (E)-N′-(3,5-dimethoxybenzylidene)-3-(4- | |
| | methoxyphenol)-1,2,4-oxadiazole-5- | |
| | carbohydrazide | |
| 76 | 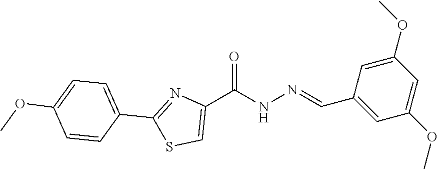 [SMILES: COc1ccc(-c2nc(C(=O)N/N=C/c3cc(OC)cc(OC)c3)cs2)cc1] | 400 MHz, DMSO-d6 δ 10.45 (s, 1H), 8.30 (s, 1H), 8.21 (s, 1H), 7.93 (d, J = 8.7 Hz, 2H), 7.01 (d, J = 8.8 Hz, 2H), 6.98 (d, J = 1.7 Hz, 2H), 6.54 (s, 1H), 3.90 (s, 3H), 3.86 (s, 6H). [M + H]+: 398.0. |
| | (E)-N′-(3,5-dimethoxybenzylidene)-2-(4- | |
| | methoxyphenyl)thiazole-4- | |
| | carbohydrazide | |

| | |
|---|---|
| 77 | 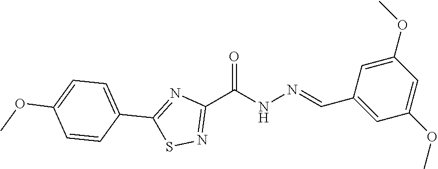 [SMILES: COc1ccc(-c2nc(C(=O)N/N=C/c3cc(OC)cc(OC)c3)ns2)cc1] |
| | (E)-N′-(3,5-dimethoxybenzylidene)-5-(4-methoxyphenyl)-1,2,4-thiadiazole-3- |
| | carbohydrazide |

### Example 2. Obtaining Compounds of Formula Ib

### Compounds 78-79

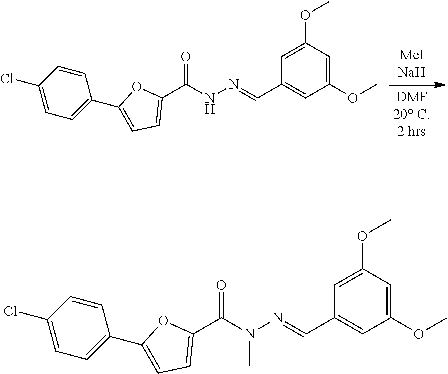 [SMILES: COc1cc(/C=N/NC(=O)c2ccc(-c3ccc(Cl)cc3)o2)cc(OC)c1 ; COc1cc(/C=N/N(C)C(=O)c2ccc(-c3ccc(Cl)cc3)o2)cc(OC)c1]

In a flask containing 3.57 mmol of the (E)-N′-(3,5-dimethoxybenzylidene)-6-(4-methoxyphenyl) pyrazine-2-carbohydrazide, dissolved in 50 mL of DMF, 4.28 mmol of NaH (60% purity) was added at 0° C. The mixture was heated at 20° C. for 2 hours. Subsequently, 9.86 mmol of methane iodide was added. Subsequently, the reaction mixture was allowed to stir at 20° C. for 2 hours. After total consumption of the starting reagent (monitored by CCF), the reaction mixture was diluted with a solution of NH4Cl. The product was filtered and used without subsequent purification. 232 mg (90% yield) of the compound of interest were obtained (Table 7).

TABLE 7
Compounds obtained, from the corresponding reagents, following
described method.

| Compound | | 1H-NMR/ |
|---|---|---|
| No. | Structure | MS m/z [M + H]+ |
| 78 | 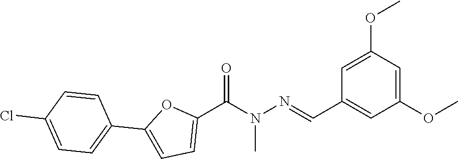 [SMILES: COc1cc(/C=N/N(C)C(=O)c2ccc(-c3ccc(Cl)cc3)o2)cc(OC)c1] | 400 MHz, DMSO-d6 δ 8.06 (1H, s), 7.82 (2H, d), 7.54 (2H, d), 7.49 (1H, d), 7.24 (1H, d), 6.90 (2H, s), 6.56 (1H, s), 3.76 (6H, s), 3.47 (3H, s). |
| | (E)-5-(4-chlorophenyl)-N′-(3,5- | |
| | dimethoxybenzylidene)-N-methylfuran-2- | |
| | carbohydrazide | |
| 79 | 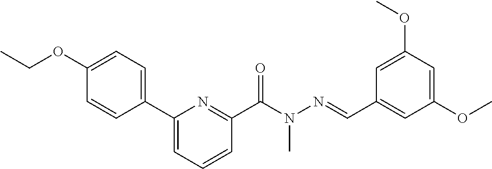 [SMILES: CCOc1ccc(-c2cccc(C(=O)N(C)/N=C/c3cc(OC)cc(OC)c3)n2)cc1] | 400 MHz, DMSO-d6 δ 8.01 (d, J = 8.8 Hz, 2H), 7.95-7.90 (m, 3H), 7.37 (dd, J = 5.5, 3.0 Hz, 1H), 6.99 (d, J = 8.9 Hz, 2H), 6.54-6.50 (m, sinais solapados, 2H), 6.40 (t, J = 2.2 Hz, 1H), 4.05 (q, J = 7.0 Hz, 2H), 3.52 (s, 6H), 3.50 (s, 3H), 1.33 (t, J = 7.0 Hz, 3H). [M + H]+: 420.4. |
| | (E)-N′-(3,5-dimethoxybenzylidene)-6-(4- | |
| | ethoxyphenyl)-N-methylpicolinohydrazide) | |

### Example 3. In Vitro Tests

Biological assays were performed on Chinese hamster ovary (CHO) cells expressing the human sodium channels Nav 1.8 and Nav 1.7 stably.

The experimental voltage-clamp protocol using the whole cell configuration was established on the automated ScreenPatch® 384P platform (SP384PE, Nanion Technologies, Livingston, NJ) and recording of the results was performed with a Nanion 384-well Patch Clamp chip (NPC) (Nanion Technologies, Livingston, NJ).

Compounds of Formula (I), were diluted in eight concentrations in the extracellular solution composed of physiological saline, buffered with HEPES (mM): NaCl, 137; KCl, 4; CaCl2, 3.8; MgCl2, 1; HEPES, 10; Glucose, 10; pH 7.4. The extracellular solution is composed of (mM) CsCl, 50; CsF, 90; MgCl2, 5; EGTA, 5; HEPES, 10; pH 7.2. The duration of exposure of each compound with the cells expressing Nav 1.7 or Nav 1.8 was at least five minutes and the assays were performed at room temperature.

The measurements of the sodium currents of Nav 1.8 and Nav 1.7 were obtained using the voltage protocols described below:
Protocol 1: A holding voltage of −90 mV was established followed by a voltage phase of 200 ms to −120 mV, followed by a voltage step of 10 mV for 1 second for Nav 1.8 or 0 mV for Nav 1.7, followed by a step of −100 mV for 20 ms, followed by a step of 20 ms for 10 mV for Nav 1.8 or 0 mV for Nav 1.7 (TP1A) before returning to the holding voltage of −90 mV.
Protocol 2: A holding voltage of −100 mV was established followed by an inactivation voltage step at −40 mV for 8 seconds, followed by a −100 mV step for ms, followed by a 20 ms step for 10 mV for Nav 1.8 or 0 mV for Nav 1.7 (TP1A) before returning to the holding voltage of −100 mV.

Protocols were repeated at a frequency of 0.1 Hz (Protocol 1) or 0.05 Hz (Protocol 2) and current amplitude was quantified over the TP1A phase log. The variation in the peak amplitude of the current was evaluated according to the Formula described below, after exposure of the cells expressing the channels, at each concentration of the different compounds:

$$\% Block = ( 1 - ( {I}_{TP 1 A , molecule} / {I}_{TP 1 A , basal} ) \times 100 \% ,$$

where |_{TP1A, basal}, and |_{TP1A, molecule} represent the entry peaks of sodium currents into TP1A prior to exposure to the compound and in the presence of the compound, respectively.

The decrease in peak current amplitude, after exposure of the cells to the compounds, was used to calculate the percent relative blocking of the channels relative to the positive control according to the Formula below:

$$\% {Block}^{′} = 100 \% - ( ( \% Block - \% CP ) * ( 100 \% / ( \% V - \% CP ) ) ,$$

where % V and % CP represent the averages of the values of current inhibition with a vehicle (DMSO) and positive controls, respectively. The sodium currents of the positive control were considered as 100%.

$$\% {Block}^{′} = ( \% 100 / [ 1 + ( [ Test ] / {IC}_{50} ) N ] ,$$

where [Test] represents the concentration of the evaluated molecule, IC₅₀ is the concentration of the compound that generates half of the maximum inhibition, N is the Hill's coefficient and % Block′ is the percentage of the sodium channel current (Nav 1.8 and Nav 1.7) inhibited at each concentration of the evaluated molecule. Data were obtained by non-linear least squares regression with XLfit for Excel (Microsoft, Redmond, WA).

Compounds were tested in at least one assay to obtain the IC₅₀ value. For compounds that were tested in two or more assays, the results are described as the average of the IC₅₀ values.

TABLE 8
IC₅₀ values for compounds selected from 1-79
in Nav 1.8 and Nav 1.7 channels, Protocol 1.

| | Nav1.8 | Nav1.7 |
|---|---|---|
| Compound No. | IC50 (nM) | IC50 (nM) |
| 1 | >30000 | >30000 |
| 2 | >30000 | >30000 |
| 3 | >30000 | >30000 |
| 4 | ND | ND |
| 5 | ND | ND |
| 6 | ND | ND |
| 7 | >6272 | >16667 |
| 8 | ND | ND |
| 9 | ND | ND |
| 10 | 1370 | 362 |
| 11 | 8580 | 11960 |
| 12 | 3210 | 1891 |
| 13 | ND | ND |
| 16 | 4245 | 4123 |
| 17 | 6825 | 7090 |
| 18 | 6825 | 7090 |
| 19 | 5899 | 5723 |
| 20 | ND | ND |
| 21 | ND | ND |
| 22 | >10000 | >10000 |
| 23 | >10000 | >10000 |
| 24 | 5899 | 5723 |
| 25 | >10000 | >10000 |
| 26 | >10000 | 3502 |
| 27 | >10000 | >10000 |
| 28 | >10000 | >10000 |
| 29 | >10000 | >10000 |
| 30 | >10000 | >10000 |
| 32 | 1361 | 1045 |
| 33 | >10000 | >10000 |
| 34 | >10000 | >10000 |
| 35 | >10000 | >10000 |
| 36 | 8425 | 7059 |
| 37 | >10000 | 8759 |
| 42 | >10000 | >10000 |
| 43 | >10000 | >10000 |
| 44 | >10000 | >10000 |
| 45 | >10000 | >10000 |
| 46 | >10000 | >10000 |
| 47 | >10000 | >10000 |
| 48 | >10000 | >10000 |
| 49 | >10000 | >10000 |
| 50 | >10000 | >10000 |
| 51 | >10000 | >10000 |
| 52 | >10000 | >10000 |
| 53 | >10000 | >10000 |
| 55 | >10000 | >10000 |
| 56 | >10000 | >10000 |
| 57 | >10000 | >10000 |
| 58 | >10000 | >10000 |
| 59 | >10000 | >10000 |
| 67 | >10000 | >10000 |
| 78 | >30000 | >30000 |
| 79 | >10000 | >10000 |

ND = IC₅₀ value not available. Percent of inhibition below 10% at a concentration of 10 μM.

TABLE 9
IC₅₀ values for selected compounds between 14-77
in Nav 1.8 and Nav 1.7 channels, Protocol 2

| | Nav1.8 | Nav1.7 |
|---|---|---|
| Compound No. | IC50 [nM] | IC50 [nM] |
| 14 | 753.6 | 1317 |
| 15 | 297.1 | 272 |
| 31 | 224.4 | 443.5 |
| 38 | >1000 | >10000 |
| 39 | >1000 | >10000 |
| 41 | >1000 | 1200 |
| 40 | 140 | 123 |
| 54 | 312.6 | 137.2 |
| 60 | 5044.1 | 5150 |
| 61 | 183.1 | 650.9 |
| 62 | 461.9 | 301.5 |
| 63 | >1000 | 305.9 |
| 64 | 383.7 | 173.1 |
| 65 | >1000 | 6441.3 |
| 66 | >1000 | 569.2 |
| 68 | 636 | >10000 |
| 69 | >1000 | >10000 |
| 71 | >1000 | >10000 |
| 72 | >1000 | >10000 |
| 73 | >1000 | >10000 |
| 74 | >1000 | >10000 |
| 75 | >1000 | >10000 |
| 76 | >1000 | >10000 |

From these results, the inhibitory activity of Nav 1.7 and/or 1.8 is proven, the application of which is readily carried out by persons skilled in the art in pharmaceutical compositions, which may comprise one or more of said compounds of Formula I, kits, in addition to uses in the treatment of pain-related pathologies.

In particular, such results indicate the possibility of using the compounds of Formula (I) in the preparation of medicaments for treating conditions such as peripheral neuropathic pain, chemotherapy-induced neuropathy, complex regional pain, neuropathy related to viral infection, neuropathy secondary to tumor infiltration, diabetic neuropathy, phantom limb pain, postherpetic neuralgia, trigeminal neuralgia, and postsurgical neuralgia.

It should be understood that the embodiments described above are merely illustrative and that various modifications may be made by a person skilled in the art thereto without departing from the scope of the present invention. Accordingly, the present invention should not be considered limited to the exemplary embodiments described in the present application.

The invention claimed is:

1. A compound characterized in that it comprises Formula (I) 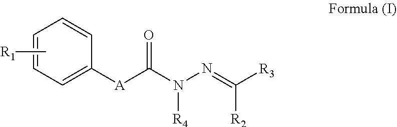
or a pharmaceutically acceptable salt, hydrate, solvate, and isomer thereof, wherein:
A is selected from the group consisting of 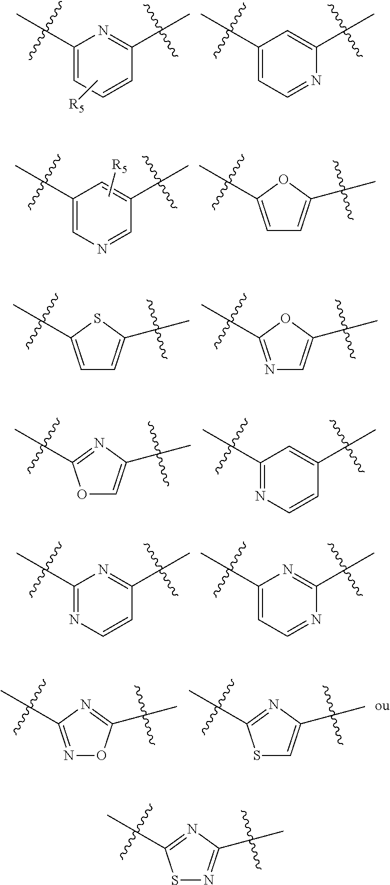
R1 is selected from the group consisting of hydrogen, halogen, linear or branched C1-6 alkoxy, 2-morpholinoethoxy, C3-6 cycloalkyloxy, or C1-5 haloalkyloxy;
R2 and R4 are independently selected from the group consisting of hydrogen or branched or linear C1-6alkyl;
R3 is selected from the group consisting of hydrogen, heterocycle or substituted heterocycle, or R6;
R5 is selected from the group consisting of hydrogen, halogen, trifluoromethyl.
R6 is 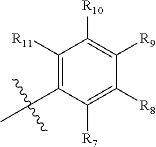
R7, R8, R9, R10 and R11 are independently selected from the group consisting of hydrogen, halogen, linear or branchedC1-6 alkoxy.

2. The compound according to claim 1, characterized in that
R1 is selected from the group consisting of hydrogen, chloro, bromo, fluoro, methoxy, ethoxy, isopropoxy, propoxy, butoxy, 2-morpholinoethoxy, cyclopropoxy; cyclopentyloxy, cyclohexyloxy, cyclobutyloxy or trifluoromethoxy;
R2 and R4 are independently selected from the group consisting of hydrogen, methyl, ethyl, propyl or isopropyl;
R3 is selected from the group consisting of hydrogen, thiophene, furan, 1H-pyrrol-2-yl, 1-methyl-1H-pyrrol-2-yl, 1-ethyl-1H-pyrrol-2-yl, 1H-imidazol-5-yl, 1H-pyrazol-5-yl, 2-oxazole, 2-thiazole, 5-oxazole, 5-thiazole, 1,3,4-oxadiazole, 1,3,4-thiadiazole, 3-isoxazole, 5-isoxazole, 3-isothiazole, 5-isothiazole, isoxazolpyridin-3-yl; pyridin-4-yl, pyridin-2-yl; 2-morpholinopyridin-3-yl, 4-((dimethylamino)methyl) thiophen-2-yl or R6;
R5 is selected from the group consisting of hydrogen, chloro, bromo, fluoro or trifluoromethyl.
R7, R8, R9, R10 and R11 are independently selected from the group consisting of hydrogen, chlorine, fluorine, bromine, methoxy, ethoxy, propoxy or isoproxy.

3. The compound of claim 1, characterized in that:
R1 is selected from the group consisting of hydrogen, chloro, bromo, fluoro, methoxy, ethoxy, isopropoxy, propoxy, butoxy, 2-morpholinoethoxy, cyclopropoxy; cyclopentyloxy, cyclohexyloxy, cyclobutyloxy or trifluoromethoxy;
R2 and R4 are independently selected from hydrogen or methyl;
R3 is selected from the group consisting of hydrogen, thiophene, pyridin-3-yl; pyridin-4-yl, pyridin-2-yl; 2-morpholinopyridin-3-yl, 4-((dimethylamino)methyl) thiophen-2-yl, or R6;
R5 is selected from the group consisting of hydrogen, chloro, bromo, fluoro or trifluoromethyl.
R7 is selected from hydrogen, chlorine, fluorine or methoxy;
R8 and R10 are independently selected from hydrogen or methoxy;
R9 is hydrogen;
R11 is selected from the group consisting of hydrogen, chlorine or fluorine.

4. The compound according to claim 1, characterized in that it is selected from the group consisting of:
(E)-5-(4-chlorophenyl)-N′-(thiophen-2-ylmethylene) furan-2-carbohydrazide (Compound 1);
(E)-5-(4-chlorophenyl)-N′-(3,5-dimethoxybenzylidene) furan-2-carbohydrazide (Compound 2);
(E)-5-(4-ethoxyphenyl)-N′-(thiophen-2-ylmethylene) furan-2-carbohydrazide (Compound 3);
(E)-5-(4-ethoxyphenyl)-N′-((2-morpholinopyridin-3-yl)methylene) furan-2-carbohydrazide (Compound 4);
(E)-N′-((4-((dimethylamino)methyl) thiophen-2-yl)methylene)-5-(4-ethoxyphenyl) furan-2-carbohydrazide (Compound 5);
(E)-5-(4-ethoxyphenyl)-N′-(pyridin-3-ylmethylene) furan-2-carbohydrazide (Compound 6);
(E)-5-(4-isopropoxyphenyl)-N′-(thiophen-2-ylmethylene) furan-2-carbohydrazide (Compound 7);
(E)-N′-((4-((dimethylamino)methyl) thiophen-2-yl)methylene)-5-(4-isopropoxyphenyl) furan-2-carbohydrazide (Compound 8);
(E)-5-(4-(2-morpholinoethoxy)phenyl)-N′-(thiophen-2-ylmethylene) furan-2-carbohydrazide (Compound 9);
(E)-6-(4-chlorophenyl)-N′-(3,5-dimethoxybenzylidene) picolinohydrazide (Compound 10);
(E)-6-(4-chlorophenyl)-N′-(thiophen-2-ylmethylene) picolinohydrazide (Compound 11);
(E)-6-(4-ethoxyphenyl)-N′-(thiophen-2-ylmethylene) picolinohydrazide (Compound 12);
(E)-6-(4-isopropoxyphenyl)-N′-(thiophen-2-ylmethylene) picolinohydrazide (Compound 13);
(E)-N′-(3,5-dimethoxybenzylidene)-6-(4-methoxyphenyl) picolinohydrazide (Compound 14);
(E)-N′-(3,5-dimethoxybenzylidene)-6-(4-propoxyphenyl) picolinohydrazide (Compound 15);
(E)-5-(4-chlorophenyl)-N′-(3,5-dimethoxybenzylidene) nicotinohydrazide (Compound 16);
(E)-N′-(3,5-dimethoxybenzylidene)-5-(4-(trifluoromethoxy)phenyl) nicotinohydrazide (Compound 17);
(E)-N′-(3,5-dimethoxybenzylidene)-5-(4-ethoxyphenyl) nicotinohydrazide (Compound 18);
(E)-N′-(3,5-dimethoxybenzylidene)-5-(4-isopropoxyphenyl) nicotinohydrazide (Compound 19);
(E)-2-(4-chlorophenyl)-N′-(3,5-dimethoxybenzylidene) isonicotinohydrazide (Compound 20);
(E)-N′-(3,5-dimethoxybenzylidene)-2-(4-(trifluoromethoxy)phenyl) isonicotinohydrazide (Compound 21);
(E)-N′-(3,5-dimethoxybenzylidene)-2-(4-ethoxyphenyl) isonicotinohydrazide (Compound 22);
(E)-N′-(3,5-dimethoxybenzylidene)-2-(4-isopropoxyphenyl) isonicotinohydrazide (Compound 23);
(E)-N′-(3,5-dimethoxybenzylidene)-6-(4-isopropoxyphenyl) picolinohydrazide (Compound 24);
(E)-N′-(3,5-dimethoxybenzylidene)-5-(4-methoxyphenyl) nicotinohydrazide (Compound 25);
(E)-N′-(3,5-dimethoxybenzylidene)-5-(4-propoxyphenyl) nicotinohydrazide (Compound 26);
(E)-5-(4-butoxyphenyl)-N′-(3,5-dimethoxybenzylidene) nicotinohydrazide (Compound 27);
(E)-5-(4-(cyclopentyloxy)phenyl)-N′-(3,5-dimethoxybenzylidene) nicotinohydrazide (Compound 28);
(E)-N′-(3,5-dimethoxybenzylidene)-2-(4-methoxyphenyl) isonicotinohydrazide (Compound 29);
(E)-N′-(3,5-dimethoxybenzylidene)-2-(4-propoxyphenyl) isonicotinohydrazide (Compound 30);
(E)-N′-(3,5-dimethoxybenzylidene)-6-(4-ethoxyphenyl) picolinohydrazide (Compound 31);
(E)-6-(4-ethoxyphenyl)-N′-(2-fluorobenzylidene) picolinohydrazide (Compound 32);
(E)-N′-(2-chlorobenzylidene)-6-(4-ethoxyphenyl) picolinohydrazide (Compound 33);
(E)-N′-(2,6-dichlorobenzylidene)-6-(4-ethoxyphenyl) picolinohydrazide (Compound 34);
(E)-N′-(2-chloro-6-fluorobenzylidene)-6-(4-ethoxyphenyl) picolinohydrazide (Compound 35);
(E)-6-(4-ethoxyphenyl)-N′-(2-methoxybenzylidene) picolinohydrazide (Compound 36);
(E)-6-(4-ethoxyphenyl)-N′-((2-morpholinopyridin-3-yl)methylene) picolinohydrazide (Compound 37);
(E)-2-(4-butoxyphenyl)-N′-(3,5-dimethoxybenzylidene) isonicotinohydrazide (Compound 38);
(E)-2-(4-(cyclopentyloxy)phenyl)-N′-(3,5-dimethoxybenzylidene) isonicotinohydrazide (Compound 39);
(E)-N′-(3,5-dimethoxybenzylidene)-6-(4-(trifluoromethoxy)phenyl) picolinohydrazide (Compound 40);
(E)-6-(4-butoxyphenyl)-N′-(3,5-dimethoxybenzylidene) picolinohydrazide (Compound 41);
(E)-4-(4-chlorophenyl)-N′-(3,5-dimethoxybenzylidene) picolinohydrazide (Compound 42);
(E)-4-(4-chlorophenyl)-N′-(thiophen-2-ylmethylene) picolinohydrazide (Compound 43);
(E)-N′-(3,5-dimethoxybenzylidene)-4-(4-ethoxyphenyl) picolinohydrazide (Compound 44);
(E)-4-(4-ethoxyphenyl)-N′-(thiophen-2-ylmethylene) picolinohydrazide (Compound 45);
(E)-N′-(3,5-dimethoxybenzylidene)-4-(4-isopropoxyphenyl) picolinohydrazide (Compound 46);
(E)-4-(4-isopropoxyphenyl)-N′-(thiophen-2-ylmethylene) picolinohydrazide (Compound 47);
(E)-4-(4-(cyclopentyloxy)phenyl)-N′-(3,5-dimethoxybenzylidene) picolinohydrazide (Compound 48);
(E)-N′-(3,5-dimethoxybenzylidene)-4-(4-(trifluoromethoxy)phenyl) picolinohydrazide (Compound 49);
(E)-4-(4-(cyclopentyloxy)phenyl)-N′-(thiophen-2-ylmethylene) picolinohydrazide (Compound 50);
(E)-N′-(thiophen-2-ylmethylene)-4-(4-(trifluoromethoxy)phenyl) picolinohydrazide (Compound 51);
(E)-2-(4-chlorophenyl)-N′-(3,5-dimethoxybenzylidene)pyrimidine-4-carbohydrazide (Compound 52);
(E)-N′-(3,5-dimethoxybenzylidene)-2-(4-ethoxyphenyl)pyrimidine-4-carbohydrazide (Compound 53);
(E)-N′-(3,5-dimethoxybenzylidene)-2-(4-isopropoxyphenyl)pyrimidine-4-carbohydrazide (Compound 54);
(E)-2-(4-(cyclopentyloxy)phenyl)-N′-(3,5-dimethoxybenzylidene)pyrimidine-4-carbohydrazide (Compound 55;
(E)-N′-(3,5-dimethoxybenzylidene)-2-(4-(trifluoromethoxy)phenyl) pyrimidine-4-carbohydrazide (Compound 56);
(E)-N′-(3,5-dimethoxybenzylidene)-2-(4-methoxyphenyl)pyrimidine-4-carbohydrazide (Compound 57);
(E)-N′-(3,5-dimethoxybenzylidene)-2-(4-propoxyphenyl)pyrimidine-4-carbohydrazide (Compound 58);
(E)-2-(4-butoxyphenyl)-N′-(3,5-dimethoxybenzylidene)pyrimidine-4-carbohydrazide (Compound 59);
(E)-4-(4-chlorophenyl)-N′-(3,5-dimethoxybenzylidene)pyrimidine-2-carbohydrazide (Compound 60);
(E)-N′-(3,5-dimethoxybenzylidene)-4-(4-ethoxyphenyl)pyrimidine-2-carbohydrazide (Compound 61);
(E)-N′-(3,5-dimethoxybenzylidene)-4-(4-isopropoxyphenyl)pyrimidine-2-carbohydrazide (Compound 62);
(E)-4-(4-(cyclopentyloxy)phenyl)-N′-(3,5-dimethoxybenzylidene)pyrimidine-2-carbohydrazide (Compound 63);
(E)-N′-(3,5-dimethoxybenzylidene)-4-(4-(trifluoromethoxy)phenyl) pyrimidine-2-carbohydrazide (Compound 64);
(E)-N′-(3,5-dimethoxybenzylidene)-4-(4-methoxyphenyl)pyrimidine-2-carbohydrazide (Compound 65);
(E)-N′-(3,5-dimethoxybenzylidene)-4-(4-propoxyphenyl)pyrimidine-2-carbohydrazide (Compound 66);
(E)-4-(4-butoxyphenyl)-N′-(3,5-dimethoxybenzylidene)pyrimidine-2-carbohydrazide (Compound 67);
(E)-N′-(3,5-dimethoxybenzylidene)-6-(4-ethoxyphenyl)-4-fluoropicolinohydrazide (Compound 68);
(E)-N′-(3,5-dimethoxybenzylidene)-6-(4-ethoxyphenyl)-4-trifluoromethyl) picolinohydrazide (Compound 69);
(E)-N′-(3,5-dimethoxybenzylidene)-5-(4-ethoxyphenyl)-4-fluoronicotinohydrazide (Compound 70);
(E)-N′-(3,5-dimethoxybenzylidene)-5-(4-methoxyphenyl) furan-2-carbohydrazide (Compound 71);
(E)-N′-(3,5-dimethoxybenzylidene)-5-(4-methoxyphenyl)thiophene-2-carbohydrazide (Compound 72);
(E)-N′-(3,5-dimethoxybenzylidene)-2-(4-methoxyphenyl) oxazole-5-carbohydrazide (Compound 73);
(E)-N′-(3,5-dimethoxybenzylidene)-2-(4-methoxyphenol) oxazole-4-carbohydrazide (Compound 74);
(E)-N′-(3,5-dimethoxybenzylidene)-3-(4-methoxyphenol)-1,2,4-oxadiazole-5-carbohydrazide (Compound 75);
(E)-N′-(3,5-dimethoxybenzylidene)-2-(4-methoxyphenyl) thiazole-4-carbohydrazide (Compound 76);
(E)-N′-(3,5-dimethoxybenzylidene)-5-(4-methoxyphenyl)-1,2,4-thiadiazole-3-carbohydrazide (Compound 77);
(E)-5-(4-chlorophenyl)-N′-(3,5-dimethoxybenzylidene)-N′-methylfuran-2-carbohydrazide (Compound 78);
(E)-N′-(3,5-dimethoxybenzylidene)-6-(4-ethoxyphenyl)-N′-methylpicolinohydrazide (Compound 79).

5. The compound according to claim 1, characterized in that it is a selective inhibitor of the voltage-gated sodium channels Nav 1.7 and/or Nav 1.8.

6. A pharmaceutical composition characterized in that it comprises a therapeutically effective amount of one or more compounds of Formula (I) or a pharmaceutically acceptable salt, hydrate, solvate and isomer thereof, as defined in claim 1; and one or more pharmaceutically acceptable excipients.

7. The pharmaceutical composition according to claim 6, characterized in that it is formulated as an oral, sublingual, nasal, parenteral, injectable, submuscular, topical, transdermal, ocular or rectal composition.

8. Use of compound(s) of Formula (I), as defined in claim 1, characterized in that it is for preparing a medicament for treating pathologies related to neuropathic pain.

9. The use according to claim 8, characterized in that said pathologies are selected from the group consisting of peripheral neuropathic pain, chemotherapy-induced neuropathy, complex regional pain, neuropathy related to viral infection, neuropathy secondary to tumor infiltration, diabetic neuropathy, phantom limb pain, postherpetic neuralgia, trigeminal neuralgia and postsurgical neuralgia.

10. A method of treating, preventing, alleviating, suppressing, and/or controlling neuropathic pain-related pathologies characterized by administering an effective amount of at least one compound of Formula (I) or a pharmaceutically acceptable salt, hydrate, solvate, and isomer thereof as defined in claim 1.

11. The method of claim 10 characterized in that it is for the treatment or prophylaxis of peripheral neuropathic pain, chemotherapy-induced neuropathy, complex regional pain, neuropathy related to viral infection, neuropathy secondary to tumor infiltration, diabetic neuropathy, phantom limb pain, postherpetic neuralgia, trigeminal neuralgia, and postsurgical neuralgia.

12. The method according to claim 10, characterised in administration of the at least one compound of Formula (I) is selected from the group comprising orally, sublingually, nasally, parenterally, injectable, submuscularly, topically, transdermally, ocularly, and rectally.

13. A process for obtaining a compound of Formula (I), as defined in claim 1, characterized in that it comprises the steps:
(a) Forming the intermediate of Formula III: 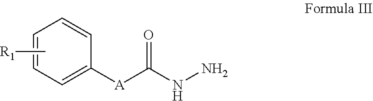
from the hydrazinolysis reaction of an intermediate of Formula IV: 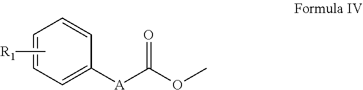
(b) obtaining a Formula I compound wherein R4 is hydrogen; 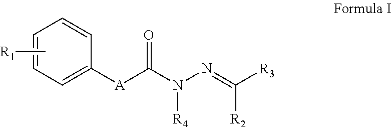
from condensation of intermediates of Formula II: 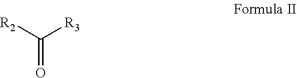
and Formula III, with or without presence of catalyst and a suitable solvent;
wherein:
A is selected from the group consisting of 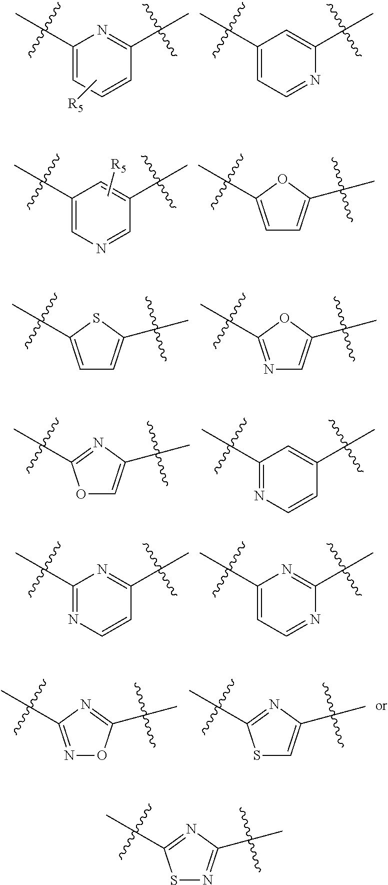
R1 is selected from the group consisting of hydrogen, halogen, linear or branched C1-6 alkoxy, 2-morpholinoethoxy, C3-6 cycloalkyloxy, or C1-5 haloalkyloxy;
R2 and R4 are independently selected from the group consisting of hydrogen or branched or linear C1-6 alkyl;
R3 is selected from the group consisting of hydrogen, heterocycle or substituted heterocycle, or R6;
R5 is selected from the group consisting of hydrogen, halogen, trifluoromethyl.
R6 is 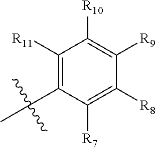
R7, R8, R9, R10 and R11 are independently selected from the group consisting of hydrogen, halogen, linear or branchedC1-6 alkoxy.

14. The process of claim 13, further characterised in that it further comprises a step (c) of forming a compound of Formula (I) wherein R4 is linear or branched C1-5 alkyl from the nucleophilic substitution reaction of a compound obtained in step (b), with linear or branched C1-6 alkyl halides in the presence of inorganic base and polar aprotic solvents.

15. The process, according to claim 13, characterized in that in step (b) said catalyst is selected from concentrated hydrochloric acid, acetic acid, trifluoroacetic acid or formic acid and said solvent is selected from dimethylformamide, dimethylsulfoxide, alcohols, or combinations thereof.

16. The process according to claim 13, characterized in that in step (c) said inorganic base is selected from K2CO3 or NaH.

17. A kit characterized in that it comprises a pharmaceutical composition as defined in claim 6; and an application device.
